(19) United States
(12) Patent Application Publication
(10) Pub. No.: US 2012/0040916 A1
(43) Pub. Date: Feb. 16, 2012

(54) MOLECULAR INHIBITORS OF THE WNT/BETA-CATENIN PATHWAY

(75) Inventors: Randall T. Moon, Kenmore, WA (US); Travis L. Biechele, Seattle, WA (US); Nathan D. Camp, Seattle, WA (US); Stephen Haggarty, Dorchester, MA (US); Daniel Fass, Winchester, MA (US)

(73) Assignees: MASSACHUSETTS INSTITUTE OF TECHNOLOGY, Cambridge, MA (US); UNIVERSITY OF WASHINGTON, Seattle, WA (US)

(21) Appl. No.: 13/141,442

(22) PCT Filed: Dec. 21, 2009
(86) PCT No.: PCT/US09/68995

§ 371 Date: Nov. 2, 2011

Related U.S. Application Data
(60) Provisional application No. 61/139,750, filed on Dec. 22, 2008.

(51) Int. Cl. A61K 38/12; A61K 31/695; A61K 31/12; A61P 43/00; A61K 31/122; A61K 31/517; A61K 31/7034; A61K 31/365; A61K 31/4375

(52) U.S. Cl. 514/21.1; 514/450; 514/63; 514/463; 514/685; 514/279; 514/680; 514/266.4; 514/25

(57) ABSTRACT

The present invention is directed toward a method of treating a subject for a condition mediated by the Wnt/β-catenin pathway by selecting a subject with a condition mediated by the Wnt/β-catenin pathway and administering to the selected subject a compound selected from the group consisting of those set forth in Table 1, Table 2, and a pharmaceutically acceptable salt thereof. A method of similarly inhibiting the Wnt/β-catenin pathway in a subject is also disclosed.

This application claims benefit of U.S. Provisional Patent Application Ser. No. 61/139,750, filed Dec. 22, 2008, which is hereby incorporated by reference in its entirety.

### FIELD OF THE INVENTION

This invention relates to molecular inhibitors of the Wnt/β-catenin pathway.

### BACKGROUND OF THE INVENTION

Wnt/β-catenin signaling regulates cell fate and proliferation during development, homeostasis, and disease. The canonical Wnt pathway describes a series of events that occur when Wnt proteins bind to cell-surface receptors of the Frizzled family, causing the receptors to activate Disheveled family proteins and ultimately resulting in a change in the amount of β-catenin that reaches the nucleus. Disheveled (DSH) is a key component of a membrane-associated Wnt receptor complex which, when activated by Wnt binding Frizzled, inhibits a second complex of proteins that includes axin, GSK-3, and the protein APC. The axin/GSK-3/APC complex normally promotes the proteolytic degradation of the β-catenin intracellular signaling molecule. After this “β-catenin destruction complex” is inhibited, a pool of cytoplasmic β-catenin stabilizes, and some β-catenin is able to enter the nucleus and interact with TCF/LEF family transcription factors to promote specific gene expression.

Numerous diseases have been linked to aberrant Wnt/β-catenin signaling and several conditions (Moon RT, “WNT and Beta-catenin Signalling: Diseases and Therapies,” Nat Rev Gen 5(9):691-701 (2004)). It is also clear that modulation of Wnt/β-catenin signaling may be therapeutic for a variety of other indications including those involving a deficit in stem/progenitor cells. Lithium chloride is currently the only FDA approved small molecule modulator of Wnt/β-catenin signaling. The narrow therapeutic range of lithium combined with the vast number of diseases linked to Wnt/β-catenin signaling begs the discovery of additional small molecule modulators.

The present invention is directed at identifying small molecule modulators of wnt/β-catenin signaling.

### SUMMARY OF THE INVENTION

One aspect of the present invention is directed toward a method of treating a subject for a condition mediated by aberrant Wnt/β-catenin signaling by selecting a subject with a condition mediated by aberrant Wnt/β-catenin signaling and administering to the selected subject a compound selected from the group consisting of those set forth in Table 1, Table 2, and a pharmaceutically acceptable salt thereof.

Another aspect of the present invention is directed toward a method of inhibiting the Wnt/P-catenin pathway in a subject including selecting a subject in need of Wnt/β-catenin pathway inhibiting and administering to the selected subject a compound selected from the group consisting of those set forth in Table 1, Table 2, and a pharmaceutically acceptable salt thereof.

### BRIEF DESCRIPTION OF THE DRAWINGS

FIGS. 1A-G illustrate that nuclear β-catenin predicts improved survival in melanoma patients and correlates with decreased tumor proliferation. FIG. 1A is a graph showing that patients with the highest levels of nuclear β-catenin (upper tertile) exhibit an increased survival probability by Kaplan-Meier analysis compared to patients in the middle and lower tertile. This trend was statistically significant by log-rank test. FIG. 1B is a graph showing metastases separated into those with the highest nuclear β-catenin levels (upper 20%, n=46) and those with lower nuclear β-catenin levels (remaining 80%, n=179). Kaplan-Meier analysis showed a significantly increased survival probability in patients with the highest amount of nuclear β-catenin (Gehan-Breslow-Wilcoxon test). FIG. 1C is a graph showing the subset of patients with available data on tumor depth (Breslow thickness) analyzed by Kaplan-Meier survival curves. Tumors were grouped based on the AJCC tumor staging guidelines for tumor depth into T1 (0-1.00 mm, n=35), T2 (1.01-2.00 mm, n=26), T3 (2.01-4.00 mm, n=32) or T4 (>4.00 mm, n=20). The survival curves exhibited an extremely significant trend by log-rank test. FIG. 1D and FIG. 1E are graphs showing tumors grouped by tumor staging depth evaluated for proliferation (FIG. 1D) and for expression of nuclear β-catenin (FIG. 1E). Bars show the mean and standard deviation for each group, while gray dots represent individual tumors. The horizontal dotted lines represent the mean Ki-67 and nuclear β-catenin seen for all tumors in the array. As expected, increasing tumor depth is associated with increased proliferation. By contrast, levels of nuclear β-catenin decrease with increasing tumor depth, suggesting that activation of Wnt/β-catenin signaling is lost with melanoma progression. The trend for both % Ki-67 and nuclear β-catenin was extremely significant by ANOVA (*p<0.002). FIG. 1F is a histogram showing primary tumors stratified into tertiles based on levels of nuclear β-catenin (see FIG. 5), and the distribution of proliferation as measured by % Ki-67 was assessed in each tertile. Patients with the highest levels of nuclear β-catenin (upper tertile, n=39) showed a lower mean % Ki-67 than patients in the middle tertile (n=39) or the lower tertile (n=40). This trend was extremely significant by ANOVA (*p<0.0001). The histogram illustrates that tumors with the lowest levels of nuclear β-catenin (lower tertile) show a clear shift towards higher proliferation compared to patients with the highest levels of nuclear β-catenin (upper tertile). FIG. 1G is a graph showing normalized levels of nuclear β-catenin in primary tumors plotted against proliferation as measured by % Ki-67, and a Deming regression analysis (diagonal line) reveals an extremely significant inverse correlation between levels of nuclear β-catenin and proliferation as measured by Ki-67 (slope=−1.089+/−0.24).

FIG. 2A-E illustrate that elevation of melanocyte differentiation markers by WNT3A corresponds with decreased tumor growth and metastasis in vivo. FIG. 2A is a heatmap of whole genome expression profiles of WNT3A or WNT5A cell lines compared to gene expression in GFP cells, which served as the reference sample. Three biologic replicates were analyzed for each cell line. The heatmap illustrates the differences between the most significant regulated genes in WNT3A cells compared to WNT5A cells by unpaired t-test. Genes that were among the most significantly regulated in WNT3A cells are listed with normalized fold-change (log2) compared to GFP cells shown in parentheses. The most significantly regulated genes include known Wnt/β-catenin targets, genes involved in melanocyte and neural crest differentiation, and genes implicated in melanoma prognosis or therapeutics. FIG. 2B is a histogram showing several genes selected for validation using real-time quantitative PCR (qPCR), including genes implicated in melanocyte differentiation (Met, Kit, Sox9, Mitf, Si/Gp100), melanoma biology (Trpm1, Kit, Mme, Mlze), and genes that are known Wnt target genes (Axin2, Met, Sox9). Genes that were upregulated in WNT3A cells by transcriptional profiling are all upregulated by qPCR, while genes that are downregulated in WNT3A cells on the array (Mlze, Mme) are also downregulated by qPCR. Genes upregulated in WNT3A cells are universally downregulated in the WNT5A cells, providing evidence that WNT5A can antagonize transcription of Wnt/β-catenin gene targets in melanoma cells, even in the absence of WNT3A. Data are expressed as log2-transformed fold-change compared to B16:GFP cells, and are representative of three or more experiments with similar results. FIG. 2C is a histogram showing gene changes induced by WNT3A inhibited upon treatment with β-catenin siRNA (20 nM) compared to control siRNA (20 nM). Data are expressed as log2-transformed fold-change in cells treated with β-catenin siRNA compared to control siRNA. FIG. 2D is a graph showing tumor explants demonstrating that B16 cells expressing WNT3A form smaller tumors than cells expressing GFP or WNT5A. Data are expressed as the mean and standard deviation from four mice for each tested cell line. The experiment shown is representative of four independent experiments with the same result, all involving at least four mice for each cell line tested. The decrease in tumor size with WNT3A was highly significant by ANOVA at 14 days post-implantation (*p=0.004). FIG. 2E is a plot showing metastases to the popliteal sentinel lymph node bed evaluated by Firefly luciferase assay, demonstrating significantly decreased metastases in tumors expressing WNT3A.

FIGS. 3 A-D illustrate figures related to tumor microarray analysis. FIG. 3A is a histogram depicting the distribution of nuclear β-catenin staining in the cohort of primary tumors. The bar below shows the cut-offs for the three tertiles used for analysis of survival in FIG. 1. FIG. 3B is a histogram depicting survival analysis in metastases. The upper 20% was selected based on both the population distribution and the absolute levels of nuclear-catenin, which correspond roughly with the upper tertile of the population. FIG. 3C is a plot showing levels of nuclear β-catenin compared in primary tumors and metastases/recurrences, showing a decrease in nuclear β-catenin in metastases/recurrences that approximated statistical significance using an unpaired two-tailed t-test. This data supports the hypothesis that Wnt/β-catenin signaling is lost with melanoma progression. FIG. 3D is a plot comparing % Ki-67 with another marker of proliferation, % PCNA. Deming regression analysis gave an extremely significant correlation, with a slope of 1.04 suggesting that proliferation was robustly measured by % Ki-67.

FIGS. 4A-D illustrate Wnt expression in the context of human melanoma. FIG. 4A is a table showing data from the NCBI Gene Expression Omnibus used to evaluate the expression of Wnt isoforms in benign nevi and melanoma tumors (see also Barrett et al., Nucleic Acids Res. D760-5 (2007), which is hereby incorporated by reference in its entirety). The datasets used include GDS1375 (Talantov et at, Clin. Cancer Res, 11(20):7234-42 (2005), which is hereby incorporated by reference in its entirety) and GDS1989 (Smith et al., Cancer Biol. Ther. 4(9):1018-29 (2005), which is hereby incorporated by reference in its entirety). The primary expression data is shown, and the above table summarizes the data from these two datasets. The data summarization is based on the reported ‘detection call’ of the Affymetrix data used for all three datasets, and the scale indicates the percentage of samples with ‘present’ calls on the expression of the different Wnt isoforms. In the primary data presented above, ‘absent’ calls are faded out. Scoring was as follows: 0 calls were ‘absent’ in all samples; +represents up to 25% of specimens have expression; ++ represents 25-50% of specimens have expression; +++ represents 50-75% of specimens have expression, ++++ represents 75-100% of specimens have expression. Few Wnt isoforms are expressed by melanoma tumors based on this transcriptional profiling, and only wnt3, wnt4, wnt5a and wnt6 were detected in melanomas from both gene datasets. FIG. 4B and FIG. 4C are histograms showing the human melanoma cell lines Me1375 (FIG. 4B) and UACC 1273 (FIG. 4C) were transduced with lentiviral constructs for encoding either GFP or WNT3A. Cells were counted after 3-7 days by hematocytometer and the panels above are representative of multiple experiments with similar results. The bars represent the average and standard deviation from three biologic replicates. P-values for two-tailed t-tests were statistically significant (*p<0.05). Expression of WNT3A also led to a consistent and reproducible decrease in proliferation by MTT assay. No consistent effect on proliferation was seen with expression of WNT5A, again similar to the B 16 cell lines. FIG. 4D is a histogram showing human melanoma cell lines cultured for 3-7 days in the presence of either 10 mM sodium chloride or 10 mM lithium chloride. Proliferation was measured by hematocytometer or MTT assay, and normalized to growth observed in the samples cultured in 10 mM sodium chloride. Lithium chloride inhibited proliferation in all human melanoma cell lines tested.

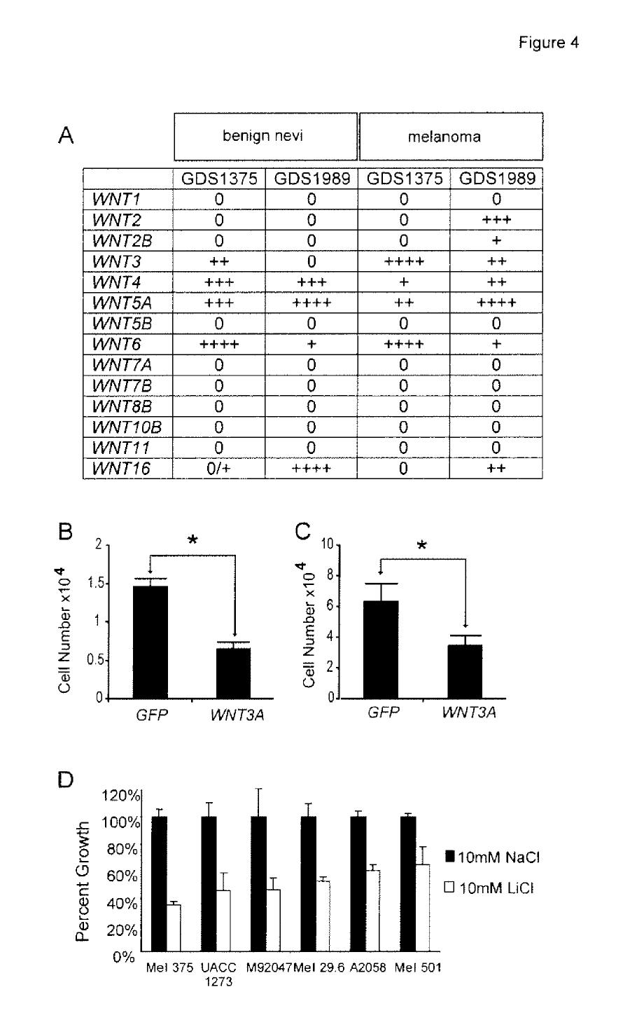

FIGS. 5A-F illustrate inhibitors of GSK3 activate Wnt/β-catenin signaling and inhibit proliferation of B16 melanoma cells. FIG. 5A and FIG. 5B are photographs showing immunofluorescent staining of β-catenin demonstrates increased nuclear β-catenin in B16 cells treated with 10 mM lithium chloride or 1 μM BIO compared to control cells treated with 10 mM sodium chloride or DMSO, respectively, consistent with activation of the Wnt/β-catenin pathway by lithium and BIO. FIG. 5C and FIG. 5D are histograms showing quantitative PCR demonstrates increased Axing levels in B 16 cells treated with 10 mM lithium chloride or 1 μM MO compared to control cells, also consistent with activation of the Wnt/β-catenin pathway by both drugs. FIG. 5E and FIG. 5F are histograms showing representative MTT proliferation assays and demonstrate the decreased proliferation seen in B16 cells treated with 10 mM lithium chloride or 1 μM BIO compared to control cells. Bars represent the mean and standard deviation of three to six biologic replicates. The difference is extremely significant by unpaired two-tailed t-test (p<0.001).

FIGS. 6A-C illustrate microarray analysis of B16 cells expressing WNT3A and WNT5A. FIG. 6A and FIG. 6B are Venn diagrams which compare the genes upregulated and downregulated in B16 cells expressing WNT3A or WNT5A compared to control B16 cells expressing GFP, which served as the reference for Agilent whole mouse genome two-channel arrays. Very few genes were regulated by WNT5A compared to WNT3A, consistent with previous results in human melanoma cells. FIG. 6C shows B16 melanoma cells transfected for 72 hours with either control siRNA or siRNA targeting murine β-catenin were analyzed by immunoblotting to assess knockdown of β-catenin protein. The siRNA sequences (SEQ ID NOs: 1-3) tested are on the right. It was found that siRNA #2 and #3 produced marked knockdown of β-catenin protein and for the validation of microarray target genes presented in FIG. 2. Cells were transfected with a pool consisting of 10 nM of siRNA #2 and #3 to minimize off-target effects of each individual siRNA.

FIG. 7 illustrates a model for differentiation therapy using Wnt/β-catenin activators in melanoma. This is a schematic diagram depicting a model of melanoma arising through transformation of differentiated melanocytes and nevus (mole) cells or from melanocytic progenitor cells, taking into account that clinical melanomas arise both from established melanocytic lesions and also de nova (Barnhill et al., Pathology of Melanocytic Nevi and Malignant Melanoma (2004), which is hereby incorporated by reference in its entirety). Based readouts of differentiation such as gene expression profiles, previous studies have found that melanoma progression appears to correlate with the loss of expression of melanocytic markers. Additionally, this model also incorporates the concept of cancer stem cells (or tumor initiating cells) in melanoma (Hendrix et al., Nat, Rev. Cancer 7:246 (2007), which is hereby incorporated by reference in its entirety), which give rise to highly proliferative bulk tumor cells, and are themselves highly resistant to conventional chemotherapy in the context of melanoma and other cancer stem cell models. Based on the finding that WNT3A is one of only three factors needed to generate functional melanocytes from embryonic stem cells (Fang et al., Stem Cells 24:1668 (2006), which is hereby incorporated by reference in its entirety), as well as the well-described requirement for Wnt/β-catenin signaling in melanocyte development from animal models (Dorsky et al., Nature 396:370 (1998), which is hereby incorporated by reference in its entirety), the leveraging of this pathway to force cell fate changes in melanoma offers an attractive choice for therapeutic manipulation. The findings herein, as well as other supporting published results (Bachmann et al., Clin. Cancer Res. 11:8606 (2005); Kageshita et al., Br. J. Dermatol. 145:210 (2001), which are hereby incorporated by reference in their entirety) documenting the loss of β-catenin with melanoma progression and decreased survival are depicted below the model.

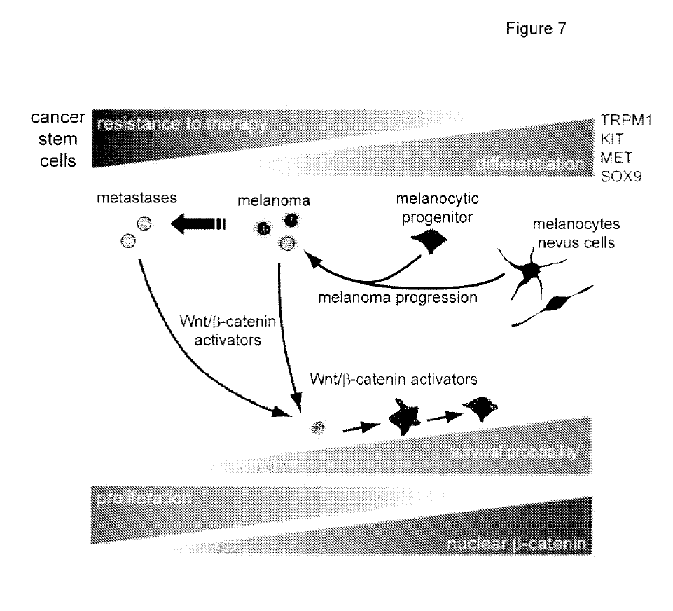

### DETAILED DESCRIPTION OF THE INVENTION

One aspect of the present invention is directed toward a method of treating a subject for a condition mediated by aberrant Wnt/β-catenin signaling by selecting a subject with a condition mediated by aberrant Wnt/β-catenin signaling and administering to the selected subject a compound selected from the group consisting of those set forth in Table 1, Table 2, and a pharmaceutically acceptable salt thereof.

In a preferred embodiment of this and other aspects described herein, the subject is human.

The condition which can be treated in accordance with this aspect of the present invention can be any one of the following: cancer (malignant melanoma, colorectal cancer, renal, liver, lung, breast, prostate, ovarian, parathyroid, leukemias, etc), bone mass diseases, fracture repair, FEVR, diabetes mellitus, cord blood transplants, psychiatric disease (e.g., bipolar depression), neurodegenerative disease (Alzheimer's, ALS), hair loss, diseases linked to loss of stem/progenitor cells, conditions improved by increasing stem/progenitor cell populations, HIV, and tooth agenesis.

Another aspect of the present invention is directed toward a method of inhibiting the Wnt/β-catenin pathway in a subject including selecting a subject in need of a Wnt/β-catenin pathway inhibiting and administering to the selected subject a compound selected from the group consisting of those set forth in Table 1, Table 2, and a pharmaceutically acceptable salt thereof.

TABLE 1
Inhibitors

| | |
|---|---|
| Family I | 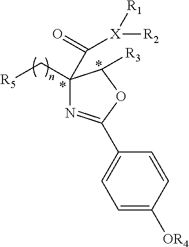 |
| | wherein: |
| | the carbon atom designated * is in the R or S configuration; and |
| | X is oxygen, nitrogen, or —CH—; |
| | n is an integer from 1 to 6; |
| | R₁ and R₂ are independently H, C₁-C₆ alkyl, C₂-C₆ alkenyl, C₂-C₆ alkynyl, |
| | C₃-C₆ cycloalkyl, C₁-C₆ alkoxy, C₂-C₁₀ alkyletheryl, or arylalkyl, C₄-C₇ |
| | cycloalkylalkyl, each optionally substituted from 1 to 3 times with |
| | substituents selected from the group consisting of phenyl, cyano, halogen, |
| | N₃, —CH₂N₃, —NH₂, and hydroxy group, wherein the phenyl group is |
| | optionally substituted from 1 to 3 times with substituents selected from the |
| | group consisting of N₃, —CH₂N₃, —NH₂, and cyano; |
| | or R₁ and R₂ can combine to form a 5- or 6-membered monocyclic |
| | heterocycle containing 1-5 heteroatoms selected from the group consisting |
| | of oxygen, nitrogen, and sulfur, optionally substituted from 1 to 4 times |
| | with substituents selected from the group consisting of H, C₁-C₆ alkyl, C₂- |
| | C₆ alkenyl, C₂-C₆ alkynyl, C₃-C₆ cycloalkyl, and C₄-C₇ cycloalkylalkyl; |
| | R₃ and R₅ are independently 5- or 6-membered monocyclic aryl or |
| | heteroaryl optionally substituted from 1 to 3 times with susbtituents selected |
| | from the group consisting of N₃, —CH₂N₃, —NH₂, hydroxy, and cyano; |
| | R₄ is independently H, C₁-C₆ alkyl, C₂-C₆ alkenyl, C₂-C₆ alkynyl, C₃-C₆ |
| | cycloalkyl, or C₄-C₇ cycloalkylalkyl, C₁-C₆ alkoxy, C₂-C₁₀ alkyletheryl, or |
| | arylalkyl, each optionally substituted from 1 to 3 times with substituents |
| | selected from the group consisting of phenyl, —OH, halogen, N₃, —CH₂N₃, |
| | —NH₂, and cyano group; |
| | with the proviso that when X is oxygen, one of R₁ or R₂ is absent |
| Family II | 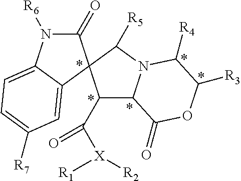 |
| | wherein: |
| | carbon atoms designated * are independently in the R or S configuration; |
| | and |
| | X is oxygen or nitrogen; |
| | R₁ and R₂ are independently H, C₁-C₆ alkyl, C₂-C₆ alkenyl, C₂-C₆ alkynyl, |
| | C₃-C₆ cycloalkyl, or C₄-C₇ cycloalkylalkyl, and arylalkyl, each optionally |
| | substituted from 1 to 3 times with substituents selected from the group |
| | consisting of phenyl, cyano, halogen, —NH₂, and hydroxy group, wherein the |
| | phenyl group is optionally substituted from 1 to 3 times with substituents |
| | selected from the group consisting of N₃, —CH₂N₃, —NH₂, and cyano; |
| | or R₁ and R₂ can combine to form a 3- to 10-membered monocyclic |
| | heterocycle containing 1-5 heteroatoms selected from the group consisting |
| | of oxygen, nitrogen, and sulfur, optionally substituted from 1 to 3 times |
| | with substituents selected from the group consisting of H, C₁-C₆ alkyl, C₂- |
| | C₆ alkenyl, C₂-C₆ alkynyl, C₃-C₆ cycloalkyl, C₄-C₇ cycloalkylalkyl, |
| | monocyclic aryl, and monocyclic heteroaryl wherein the monocyclic aryl or |
| | heteroaryl containing 1-5 heteroatoms selected from the group consisting of |
| | oxygen, nitrogen, and sulfur, and each of the monocyclic aryl or heteroaryl |
| | is optionally substituted with substitutents selected from the group |
| | consisting of halogen, hydroxy, NH₂, and cyano; |
| | R₃-R₄ are independently 5- or 6-membered monocyclic aryl or heteroaryl |
| | optionally substituted with substituents selected from the group consisting |
| | of hydroxy, halogen, —NH₂, cyano, C₁-C₆ alkyl, C₂-C₆ alkenyl, C₂-C₆ |
| | alkynyl, C₃-C₆ cycloalkyl, C₄-C₇ cycloalkylalkyl, C₁-C₆ alkoxycarbonyl, C₁- |
| | C₆ alkoxycarboxyl, C₁-C₆ alkoxycarbamoyl, and —OR₈ each optionally |
| | substituted from 1 to 3 times with substituents selected from the group |
| | consisting of C₁-C₆ alkyl, C₁-C₆ alkenyl, C₁-C₆ alkynyl, phenyl, and |
| | hydroxyphenyl, wherein R₈ is defined as below; |
| | R₆ is independently H, C₁-C₆ alkyl, C₂-C₆ alkenyl, C₂-C₆ alkynyl, C₃-C₆ |
| | cycloalkyl, C₄-C₇ cycloalkylalkyl, R₉O—(CH₂)ₙ—OC(O)—, or R₉—OC(O)— |
| | wherein n is an integer from 1 to 6 R₉ is defined as below; |
| | R₇ is independently H, C₁-C₆ alkyl, C₂-C₆ alkenyl, C₂-C₆ alkynyl, C₃-C₆ |
| | cycloalkyl, or C₄-C₇ cycloalkylalkyl, each optionally substituted from 1 to 3 |
| | times with substituents selected from the group consisting of phenyl, C₃-C₁₀ |
| | cycloalkyl, C₄-C₁₁ cycloalkylalkyl, C₃-C₁₀ cycloalkenyl, and C₄-C₁₁ |
| | cycloalkenylalkyl, each optionally substituted from 1 to 3 times with |
| | substitutents selected from the group consisiting of C₁-C₆ alkyl, C₂-C₆ |
| | alkenyl, C₂-C₆ alkynyl, C₃-C₁₀ cycloalkyl, C₄-C₁₁ cycloalkylalkyl, C₃-C₁₀ |
| | cycloalkenyl, C₃-C₁₀ cycloalkenylalkyl, C₁-C₆ alkoxy, C₁-C₆ alkoxyetheryl, |
| | C₁-C₆ alkoxycarboxyl, C₁-C₆ alkoxycarboxyl, —OH, halogen, —NH₂, and |
| | cyano; |
| | R₈ is independently H, C₁-C₆ alkyl, C₂-C₆ alkenyl, C₂-C₆ alkynyl, C₃-C₆ |
| | cycloalkyl, C₄-C₇ cycloalkylalkyl, C₁-C₆ alkoxy, C₁-C₆ alkoxy carboxyl, C₁- |
| | C₆ alkoxy carbamoyl; |
| | R₉ is independently H, C₁-C₆ alkyl, C₂-C₆ alkenyl, C₂-C₆ alkynyl, C₃-C₆ |
| | cycloalkyl, C₄-C₇ cycloalkylalkyl; |
| | with the proviso that when X is oxygen, one of R₁ or R₂ is absent. |
| Family III | 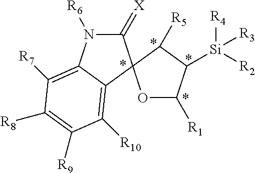 |
| | wherein: |
| | carbon atoms designated * are independently in the R or S configuration; |
| | and |
| | X is optionally oxygen, —CH₂—, or sulfur; |
| | R₁-R₃ and R₅ are independently H, C₁-C₆ alkyl, C₂-C₆ alkenyl, C₂-C₆ |
| | alkynyl, C₃-C₆ cycloalkyl, C₄-C₇ cycloalkylalkyl, C₁-C₆ alkoxy, or C₁-C₆ |
| | amidoalkyl, each optionally substituted from 1 to 3 times with substituents |
| | selected from the group consisting of monocyclic aryl, monocyclic |
| | heteroaryl, arylalkyl, cyano, halogen, —NH₂, and hydroxy group, wherein the |
| | monocyclic aryl or heteroaryl containing 1-5 heteroatoms selected from the |
| | group consisting of nitrogen, sulfur, and oxygen, is optionally substituted |
| | from 1 to 3 times with substituents selected from the group consisting of C₁- |
| | C₆ alkyl, C₂-C₆ alkenyl, C₂-C₆ alkynyl, C₃-C₆ cycloalkyl, C₄-C₇ |
| | cycloalkylalkyl, C₁-C₆ alkoxy, arylalkoxy, and arylalkyl, each optionally |
| | substituted with substituents selected from the group consisting of halogen, |
| | hydroxy, —NH₂, and cyano; |
| | R₄ is a monocyclic aryl optionally substituted with substituents selected |
| | from the group consisting of halogen, hydroxy, —NH₂, cyano, C₁-C₆ alkyl, |
| | C₂-C₆ alkenyl, C₇-C₆ alkynyl, C₃-C₆ cycloalkyl, C₄-C₇ cycloalkylalkyl, C₁- |
| | C₆ alkoxy, and C₁-C₆ alkoxyetheryl; |
| | R₅-R₇ are independently halogen, H, C₁-C₆ alkyl, C₂-C₆ alkenyl, C₂-C₆ |
| | alkynyl, C₃-C₆ cycloalkyl, C₄-C₇ cycloalkylalkyl, or arylalkyl, each |
| | optionally substituted from 1 to 3 times with substituents selected from the |
| | group consisting of phenyl, cyclocarbamoyl, cyano, halogen, —NH₂, |
| | hydroxy, and R₁₂C(O)N(R₁₁)— group, wherein the phenyl group is optionally |
| | substituted with substituents selected from the group consisting of halogen, |
| | hydroxy, —NH₂, and cyano, wherein R₁₁ and R₁₂ are defined below; |
| | or R₆ and R₇ can combine to form a fused 5 or 6-membered monocyclic |
| | heterocycle or heteroaryl containing 1-5 heteroatoms selected from the |
| | group consisting of oxygen, nitrogen, and sulfur, optionally substituted with |
| | substituents selected from the group consisting of C₁-C₆ alkyl, C₂-C₆ |
| | alkenyl, C₂-C₆ alkynyl, C₃-C₆ cycloalkyl, C₄-C₇ cycloalkylalkyl, |
| | monocyclic aryl, and monocyclic heteroaryl, wherein the heteroaryl |
| | containing 1-5 heteroatoms selected from the group consisting of oxygen, |
| | nitrogen, and sulfur, is optionally substituted with substitutents selected |
| | from the group consisting of halogen, hydroxy, and cyano; |
| | R₈-R₁₀ are independently halogen, hydroxy, —NH₂, cyano, H, C₁-C₆ alkyl, |
| | C₂-C₆ alkenyl, C₂-C₆ alkynyl, C₃-C₆ cycloalkyl, C₄-C₇ cycloalkylalkyl, C₁- |
| | C₆ alkoxy, C₁-C₆ alkoxyetheryl, |
| | C₁-C₆ amidoalkyl, 4- to 6-membered lactam, and C₃-C₆ cyclocarbamoyl, |
| | each optionally substituted from 1 to 3 times with substituents selected from |
| | the group consisting of halogen, hydroxy, —NH₂, and cyano. |
| | R₁₁-R₁₂ are independently H, C₁-C₆ alkyl, C₂-C₆ alkenyl, C₂-C₆ alkynyl, C₃- |
| | C₆ cycloalkyl, C₄-C₇ cycloalkylalkyl, C₁-C₆ alkoxy, 5- to 6-monocyclic |
| | aryl, or arylakyl; each optionally substituted from 1 to 3 times with |
| | substituents selected from the group consisting of H, C₁-C₆ alkyl, C₂-C₆ |
| | alkenyl, C₂-C₆ alkynyl, C₃-C₆ cycloalkyl, C₄-C₇ cycloalkylalkyl, C₁-C₆ |
| | alkoxy, halogen, hydroxy, —NH₂, and cyano. |
| Family IV | 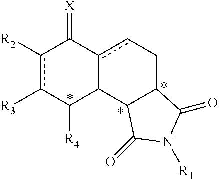 |
| | wherein: |
| | carbon atoms designated * are independently in the R or S configuration; |
| | and |
| | [symbol] represents an optional double bond; |
| | X is oxygen or sulfur; |
| | R₁-R₄ are independently halogen, hydroxy, —NH₂, cyano, H, or alkyl aryl, |
| | C₁-C₆ alkyl, C₂-C₆ alkenyl, C₂-C₆ alkynyl, C₃-C₆ cycloalkyl, or C₄-C₇ |
| | cycloalkylalkyl, or aryl alkyl each optionally substituted from 1 to 3 times |
| | with substituents selected from the group consisting of phenyl, cyano, |
| | halogen, —NH₂, and hydroxy group, wherein said phenyl group is optionally |
| | substituted with substituents selected from the group consisting of halogen, |
| | hydroxy, —NH₂, and cyano; |
| | or R₂ and R₃ can combine to form a fused 3 to 6-membered monocyclic |
| | heterocycle containing 1-5 heteroatoms selected from the group consisting |
| | of oxygen, nitrogen, and sulfur, optionally substituted with substituents |
| | selected from the group consisting of H, C₁-C₆ alkyl, C₂-C₆ alkenyl, C₂-C₆ |
| | alkynyl, C₃-C₆ cycloalkyl, C₄-C₇ cycloalkylalkyl, monocyclic aryl, and |
| | monocyclic heteroaryl wherein the heteroaryl containing 1-5 heteroatoms |
| | selected from the group consisting of oxygen, nitrogen, and sulfur, is |
| | optionally substituted with substitutents selected from the group consisting |
| | of halogen, hydroxy, and cyano. |
| Family V | 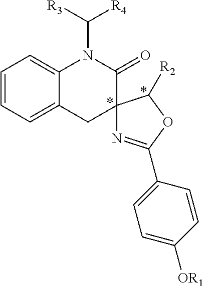 |
| | wherein: |
| | carbon atoms designated * are independently in the R or S configuration; |
| | and |
| | R₁ is H, C₁-C₆ alkyl, C₂-C₆ alkenyl, C₂-C₆ alkynyl, C₃-C₆ cycloalkyl, or C₄- |
| | C₇ cycloalkylalkyl, each optionally substituted from 1 to 3 times with |
| | substituents selected from the group consisting of, cyano, halogen, —NH₂, |
| | and hydroxy group; |
| | R₂ is a monocyclic aryl optionally substituted from 1 to 4 times with |
| | substituents selected from the group consisting of halogen, hydroxy, —NH₂, |
| | cyano, C₁-C₆ alkyl, C₂-C₆ alkenyl, C₂-C₆ alkynyl, C₃-C₆ cycloalkyl, or C₄- |
| | C₇ cycloalkylalkyl, C₁-C₆ alkoxy, and C₁-C₆ alkoxyetheryl; |
| | R₃ is H, C₁-C₆ alkyl, C₂-C₆ alkenyl, C₂-C₆ alkynyl, C₃-C₆ cycloalkyl, C₄-C₇ |
| | cycloalkylalkyl, 5- to 6-memebered monocyclic aryl, or heteroaryl, wherein |
| | the heteroaryl contains 1-5 heteroatoms selected from the group consisting |
| | of oxygen, nitrogen, and sulfur, and each of R₃ is optionally substituted |
| | from 1 to 3 with substitutents selected from the group consisiting of |
| | halogen, hydroxy, cyano, —NH₂, R₅SO₂—, R₅SO—, R₅S—, and R₅C(O)—, |
| | wherein R₅ is defined below; |
| | R₄ is H or ═O; |
| | R₅ is a 5 or 6-membered monocyclic aryl or heterocycle each optionally |
| | substituted from 1 to 3 times with susbtituents selected from the group |
| | consisting of halogen, hydroxy, cyano, and —NH₂. |
| Family VI | 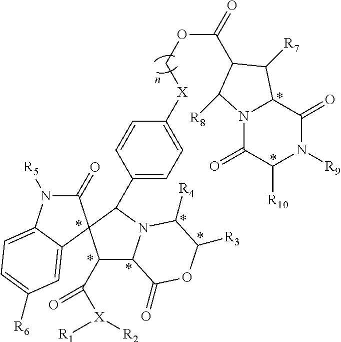 |
| | wherein: |
| | carbon atoms designated * are independently in the R or S configuration; |
| | and |
| | X is oxygen, sulfur, nitrogen, or —CH₂—; |
| | n is an interger from 1 to 6; |
| | R₁ and R₂ are independently H, C₁-C₆ alkyl, C₂-C₆ alkenyl, C₂-C₆ alkynyl, |
| | C₃-C₆ cycloalkyl, or C₄-C₇ cycloalkylalkyl, each optionally substituted from |
| | 1 to 3 times with substituents selected from the group consisting of phenyl, |
| | cyano, halogen, —NH₂, and hydroxy group, wherein the phenyl group is |
| | optionally substituted with substituents selected from the group consisting |
| | of N₃, —CH₂N₃, —NH₂, and cyano; |
| | or R₁ and R₂ can combine to form a 3- to 10-membered monocyclic |
| | heterocycle containing 1-5 heteroatoms selected from the group consisting |
| | of oxygen, nitrogen, and sulfur, optionally substituted with substituents |
| | selected from the group consisting of H, C₁-C₆ alkyl, C₂-C₆ alkenyl, C₂-C₆ |
| | alkynyl, C₃-C₆ cycloalkyl, C₄-C₇ cycloalkylalkyl, monocyclic aryl, and |
| | monocyclic heteroaryl wherein the heteroaryl containing 1-5 heteroatoms |
| | selected from the group consisting of oxygen, nitrogen, and sulfur, is |
| | optionally substituted with substitutents selected from the group consisting |
| | of halogen, hydroxy, NH₂, and cyano; |
| | R₃-R₄ and R₈ are independently 5 or 6-membered monocyclic aryl or |
| | heteroaryl each optionally substituted from 1 to 3 times with susbtituents |
| | selected from the group consisting of hydroxy, halogen, —NH₂, cyano, C₁-C₆ |
| | alkyl, C₂-C₆ alkenyl, C₂-C₆ alkynyl, C₃-C₆ cycloalkyl, C₄-C₇ |
| | cycloalkylalkyl, C₁-C₆ alkoxycarbonyl, C₁-C₆ alkoxycarboxyl, and C₁-C₆ |
| | alkoxycarbamoyl, R₁₂O—(CH₂)ₙ—O—; |
| | R₅-R₆ and R₇ are independently halogen, H, C₁-C₆ alkyl, C₂-C₆ alkenyl, C₂- |
| | C₆ alkynyl, C₃-C₆ cycloalkyl, or C₄-C₇ cycloalkylalkyl; |
| | R₉ and R₁₀ are independently H, C₁-C₁₀ alkyl, C₂-C₁₀ alkenyl, C₂-C₁₀ |
| | alkynyl, C₃-C₁₀ cycloalkyl, or C₄-C₁₁ cycloalkylalkyl, C₁-C₁₀ |
| | alkoxycarbonyl, C₁-C₁₀ alkoxycarboxyl, or C₁-C₁₀ alkoxycarbamoyl, each |
| | optionally substituted from 1 to 3 times with substituents selected from the |
| | group consisting of cyano, halogen, —NH₂, R₁₁C(O)NH—, —C(O)H, —COOH, |
| | R₁₁C(O)—, —NR₁₁R₁₂, and hydroxy group wherein R₁₁ and R₁₂ are defined |
| | below; |
| | or R₉ and R₁₀ can combine to form a 3 to 10-membered monocyclic |
| | heterocycle containing 1-5 heteroatoms selected from the group consisting |
| | of oxygen, nitrogen, and sulfur, optionally substituted with substituents |
| | selected from the group consisting of H, C₁-C₆ alkyl, C₂-C₆ alkenyl, C₂-C₆ |
| | alkynyl, C₃-C₆ cycloalkyl, C₄-C₇ cycloalkylalkyl, monocyclic aryl, and |
| | monocyclic heteroaryl wherein the monocyclic aryl or heteroaryl containing |
| | 1-5 heteroatoms selected from the group consisting of oxygen, nitrogen, and |
| | sulfur is optionally substituted with substitutents selected from the group |
| | consisiting of halogen, hydroxy, NH₂, and cyano; |
| | R₁₁-R₁₂ is independently H, C₁-C₆ alkyl, C₂-C₆ alkenyl, C₂-C₆ alkynyl, C₃- |
| | C₆ cycloalkyl, C₄-C₇ cycloalkylalkyl; |
| | with the proviso that when X is oxygen or sulfur, one of R₁ or R₂ is absent. |
| Family VII | 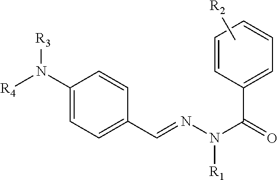 |
| | wherein: |
| | R₁-R₄ are independently H, C₁-C₆ alkyl, C₂-C₆ alkenyl, C₂-C₆ alkynyl, C₃- |
| | C₆ cycloalkyl, or C₄-C₇ cycloalkylalkyl 5- to 6-membered monocyclic aryl |
| | or heteroaryl containing 1 to 5 heteroatoms selected from the group |
| | consisting of oxygen, nitrogen, and sulfur, each of R₁-R₄ optionally |
| | substituted from 1 to 3 times with substituents selected from the group |
| | consisting of cyano, halogen, N₃, —CH₂N₃, —NH₂, —COOH, —C(O)NH₂, |
| | —C(O)NHOH, and hydroxy group. |
| Family VIII | 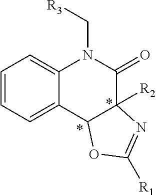 |
| | wherein: |
| | carbon atoms designated * are independently in the R or S configuration; |
| | and |
| | R₁ is a monocyclic aryl optionally substituted with substituents selected |
| | from the group consisting of halogen, hydroxy, —NH₂, cyano, C₁-C₆ alkyl, |
| | C₂-C₆ alkenyl, C₂-C₆ alkynyl, C₃-C₆ cycloalkyl, C₄-C₇ cycloalkylalkyl, C₁- |
| | C₆ alkoxyetheryl, and —OR₄, wherein R₄ is defined as below; |
| | R₂ is independently H, C₁-C₆ alkyl, C₂-C₆ alkenyl, C₂-C₆ alkynyl, C₃-C₆ |
| | cycloalkyl, C₄-C₇ cycloalkylalkyl, or arylalkyl, each optionally substituted |
| | from 1 to 3 times with substituents selected from the group consisting of |
| | cyano, halogen, —NH₂, hydroxyaryl, R₄O—C(O)—, R₄O—, and R₄C(O)— group, |
| | wherein R₄ is defined as below; |
| | R₃ is H, C₁-C₆ alkyl, C₂-C₆ alkenyl, C₂-C₆ alkynyl, C₃-C₆ cycloalkyl, C₄-C₇ |
| | cycloalkylalkyl, or a monocyclic aryl optionally substituted from 1 to 4 |
| | times with substituents selected from the group consisting of halogen, |
| | hydroxy, —NH₂, cyano, C₁-C₆ alkyl, C₂-C₆ alkenyl, C₂-C₆ alkynyl, C₃-C₆ |
| | cycloalkyl, C₄-C₇ cycloalkylalkyl, C₁-C₆ alkoxy, and C₁-C₆ alkoxyetheryl; |
| | R₄ is independently H, C₁-C₆ alkyl, C₂-C₆ alkenyl, C₂-C₆ alkynyl, C₃-C₆ |
| | cycloalkyl, C₄-C₇ cycloalkylalkyl, and C₁-C₆ alkoxyetheryl, each optionally |
| | substituted from 1 to 3 times with substituents selected from the group |
| | consisting of H, halogen, hydroxyl, —NH₂, and cyano. |
| Family IX | 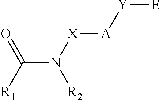 |
| | wherein: |
| | X is optionally —HC═N—, —C(NH)—, or —O—; |
| | A is optionally C₁-C₆ alkyl, C₂-C₆ alkenyl, C₂-C₆ alkynyl, C₃-C₆ cycloalkyl, |
| | C₃-C₆ cycloalkenyl, 5- to 6-membered monocyclic aryl, or heteroaryl |
| | containing 1-5 heteroatoms selected from the group consisting of oxygen, |
| | sulfur, and nitrogen, each one of A is optionally substituted with |
| | substituents selected from the group consisting of hydroxy, halogen, —NH₂, |
| | —NHR₃, —NR₃R₄, cyano, C₁-C₆ alkyl, C₂-C₆ alkenyl, C₂-C₆ alkynyl, and C₄-C₇ |
| | cycloalkylalkyl, wherein R₃ and R₄ are defined as below; |
| | Y is an optional linker selected from the group consisting of —C(O)NH—, |
| | —C(S)NH—, —C(O)NR₁—, and —C(S)NR₁—, wherein R₁ is defined below; |
| | E is optionally a monocyclic aryl, heteroaryl, C₁-C₆ alkyl, C₁-C₆ carbamoyl, |
| | C₁-C₆ alkoxy, C₁-C₆ alkenyl, or C₁-C₆ cycloalkyl, each optionally |
| | substituted with substituents selected from the group consisting of halogen, |
| | hydroxy, cyano, —NH₂, C₁-C₆ alkyl, C₂-C₆ alkenyl, C₂-C₆ alkynyl, and C₃-C₆ |
| | cycloalkyl; |
| | R₁ is optionally a 5- to 6-membered monocyclic aryl heterocycle, or |
| | heteroaryl, each substituted with substituents selected from the group |
| | consisting of halogen, hydroxy, cyano, —NH₂, C₁-C₆ alkyl, C₂-C₆ alkenyl, |
| | and C₂-C₆ alkynyl; |
| | R₂ is optionally H, C₁-C₆ alkyl, C₂-C₆ alkenyl, C₂-C₆ alkynyl, C₃-C₆ |
| | cycloalkyl, or C₄-C₇ cycloalkyl alkyl; |
| | R₂ and R₃ are independently C₁-C₆ alkyl, C₂-C₆ alkenyl, C₂-C₆ alkynyl, C₃- |
| | C₆ cycloalkyl, or C₄-C₇ cycloalkyl alkyl; |
| | R₂ and R₃ can combine to form a heterocycle containing 1-5 heteroatom |
| | selected from the group consisting of nitrogen, oxygen, and sulfur; |
| | R₄ is C₁-C₆ alkyl, C₂-C₆ alkenyl, C₂-C₆ alkynyl, C₃-C₆ cycloalkyl, or C₄-C₇ |
| | cycloalkyl alkyl. |
| Family X | 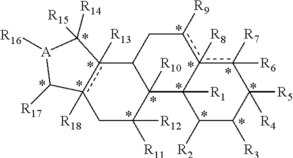 |
| | wherein: |
| | Carbon atoms designated * are independently in the R or S configuration; |
| | and |
| | [symbol] represents an optional double bond; |
| | A is a —CH— or O; |
| | R₁-R₁₈ are optionally and independently H, —OH, halogen, C₁-C₆ alkyl, C₂- |
| | C₆ alkenyl, C₂-C₆ alkynyl, C₃-C₆ cycloalkyl, C₄-C₇ cycloalkylalkyl, C₁-C₁₀ |
| | alkoxycarboxyl, C₁-C₁₀ alkoxycarbamoyl, C₁-C₁₀ alkoxycarbonyl, or C₁-C₁₀ |
| | hydroxyketoalkyl each optionally substituted from 1 to 3 times with |
| | substituents selected from the group consisting of hydroxy, —NH₂, cyano, |
| | and halogen; |
| | or R₄ and R₅ can combine to form the carbonyl group; |
| | R₁₁ and R₁₂ can combine to form the carbonyl group; |
| | R₁₄ and R₁₅ can combine to form the carbonyl group; |
| | R₅ and R₆, R₇ and R₈, R₁₀ and R₁₂, and R₁₅ and R₁₆ can combine to form |
| | independently a 3- to 6-membered heterocycle containing 1 to 5 |
| | heteroatoms selected from the group consisting of nitrogen, sulfur, and |
| | oxygen, wherein said heterocycle is optionally substituted with substituents |
| | selected from the group consisiting of H, —OH, halogen, C₁-C₆ alkyl, C₂-C₆ |
| | alkenyl, C₂-C₆ alkynyl, C₃-C₆ cycloalkyl, and C₄-C₇ cycloalkylalkyl; |
| | with the proviso that when A is oxygen R₁₆ is absent. |
| Family XI | 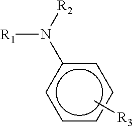 |
| | wherein: |
| | R₁-R₂ are independently H, C₁-C₆ alkyl, C₂-C₆ alkenyl, C₂-C₆ alkynyl, C₃- |
| | C₆ cycloalkyl, R₄NHC(O)—, C₄-C₇ cycloalkylalkyl, or a 5- to 6-memebered |
| | heterocycle containing 1-5 heteroatoms selected from the group consisting |
| | of oxygen, nitrogen, and sulfur, wherein each of R₁ is optionally substituted |
| | from 1 to 3 times with substitutents selected from the group consisting of |
| | cyano, halogen, —OH, —NH₂, R₄SO₂—, R₄SO—, R₄S—, C₁-C₆ alkyl, C₂-C₆ |
| | alkenyl, C₂-C₆ alkynyl, C₃-C₆ cycloalkyl, and C₄-C₇ cycloalkylalkyl, |
| | wherein R₄ is defined as below; |
| | R₃ is cyano, halogen, —OH, —NH₂, H, C₁-C₆ alkyl, C₂-C₆ alkenyl, C₂-C₆ |
| | alkynyl, C₃-C₆ cycloalkyl, or C₄-C₇ cycloalkylalkyl, each optionally |
| | substituted from 1 to 3 times with substituents selected from the group |
| | consisting of cyano, halogen, —OH, and —NH₂; |
| | R₄ is a monocyclic aryl, heterocycle, or heteroaryl containing 1-5 |
| | heteroatoms selected from the group consisting of oxygen, nitrogen, and |
| | sulfur, each of R₄ susbtituted with substituents selected from the group |
| | consisting of cyano, halogen, —OH, —NH₂, H, C₁-C₆ alkyl, C₂-C₆ alkenyl, C₂- |
| | C₆ alkynyl, C₃-C₆ cycloalkyl, and C₄-C₇ cycloalkylalkyl. |
| Family XII | 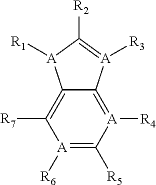 |
| | wherein: |
| | A is independently carbon, oxygen, nitrogen, or sulfur; and |
| | R₁-R₇ are independently halogen, —OH, —NH₂, —NHR₃, —NR₈R₄, H, C₁-C₆ |
| | alkyl, C₂-C₆ alkenyl, C₂-C₆ alkynyl, C₃-C₆ cycloalkyl, or C₄-C₇ |
| | cycloalkylalkyl, monocyclic aryl, monocyclic heterocyclyl, or monocyclic |
| | heteroaryl, each optionally substituted from 1 to 3 times with substituents |
| | selected from the group consisting of phenyl, C₁-C₆ alkyl, C₂-C₆ alkenyl, |
| | C₂-C₆ alkynyl, C₃-C₆ cycloalkyl, C₄-C₇ cycloalkylalkyl, cyano, halogen, |
| | —NH₂, and hydroxy group, each optionally substituted from 1 to 3 times with |
| | substituents selected from the group consisting of cyano, halogen, —NH₂, |
| | and hydroxy group, wherein R₈ and R₉ are defined below; |
| | or R₁ and R₂ or R₂ and R₃ can optionally combine to form a 3 to 10- |
| | membered monocyclic heterocycle containing 1-5 heteroatoms selected |
| | from the group consisting of oxygen, nitrogen, and sulfur, optionally |
| | substituted from 1 to 3 times with substituents selected from the group |
| | consisting of H, C₁-C₆ alkyl, C₂-C₆ alkenyl, C₂-C₆ alkynyl, C₃-C₆ |
| | cycloalkyl, C₄-C₇ cycloalkylalkyl, monocyclic aryl, R₁₀C(O)—, R₁₀C(O), |
| | R₈R₉N—C(O)—, and monocyclic heteroaryl wherein the monocyclic aryl or |
| | heteroaryl containing 1-5 heteroatoms selected from the group consisting of |
| | oxygen, nitrogen, and sulfur is optionally substituted with substituents |
| | selected from the group consisting of halogen, hydroxy, NH₂, and cyano, |
| | wherein R₈-R₁₀ are defined below; |
| | R₈-R₁₀ are independently H, C₁-C₆ alkyl, C₂-C₆ alkenyl, C₂-C₆ alkynyl, C₃- |
| | C₆ cycloalkyl, or C₄-C₇ cycloalkylalkyl, C₁-C₆ alkoxy, C₁-C₆ alkoxy |
| | carboxyl, C₁-C₆ alkoxy carboxyl, monocyclic aryl, and monocyclic |
| | heteroaryl containing 1-5 heteroatoms selected from the group consisting of |
| | oxygen, sulfur, and nitrogen, each of R₈-R₁₀ optionally substituted with |
| | halogen, hydroxyl, cyano, —NH₂, C₄-C₆ alkyl, C₂-C₆ alkenyl, C₂-C₆ alkynyl, |
| | C₃-C₆ cycloalkyl, C₄-C₇ cycloalkylalkyl, and C₁-C₆ alkoxy. |
| Family XIII | 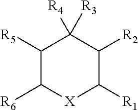 |
| | wherein: |
| | X is oxygen, sulfur, or —CH₂— |
| | R₁-R₆, are independently halogen, hydroxy, cyano, carbamoyl, —NH₂, H, C₁- |
| | C₆ alkyl, C₂-C₆ alkenyl, C₂-C₆ alkynyl, C₃-C₆ cycloalkyl, C₄-C₇ |
| | cyloalkylalkyl, C₁-C₆ alkoxy, C₁-C₆ alkoxycarbonyl, C₁-C₆ alkoxycarboxyl, |
| | or C₁-C₈ alkylalkanoate, each optionally substituted from 1 to 3 times with |
| | substituents selected from the group consisting of halogen, hydroxy, —NH₂, |
| | cyano, monocyclic aryl, monocyclic heterocyclyl, bicyclic aryl, bi-aryl, and |
| | bicyclic heteroaryl, wherein the monocyclic aryl, bi-aryl, monocyclic |
| | heterocyclyl, bicyclic aryl, or bicyclic heteroaryl containing 1-5 |
| | heteroatoms selected from the group consisting of nitrogen, sulfur, and |
| | oxygen, are each optionally substituted with substituents selected from the |
| | group consisting of halogen, hydroxy, —NH₂, H, C₁-C₆ alkyl, C₂-C₆ alkenyl, |
| | C₂-C₆ alkynyl, C₃-C₆ cycloalkyl, C₄-C₇ cyloalkylalkyl, C₁-C₆ alkoxy, and |
| | cyano; |
| | R₃ and R₄ can combine to form a carbonyl, ketal, C₄-C₆ lactone, C₄-C₆ |
| | lactame, or epoxide; |
| | R₂ and R₃ or R₄ and R₅ can independently combine to form a 3- to 12- |
| | membered monocyclic heterocycle containing 1-5 heteroatoms selected |
| | from the group consisting of oxygen, nitrogen, and sulfur, optionally |
| | substituted from 1 to 3 times with substituents selected from the group |
| | consisting of H, C₁-C₆ alkyl, C₂-C₆ alkenyl, C₂-C₆ alkynyl, C₃-C₆ |
| | cycloalkyl, C₄-C₇ cycloalkylalkyl, C₁-C₆ alkoxy, C₁-C₈ alkylalkanoate, C₁- |
| | C₆ alkylcarbamoyl, C₁-C₆ alkoxycarbamoyl, C₁-C₆ alkoxycarboxyl, C₁-C₆ |
| | alkoxycarbonyl, monocyclic aryl, and monocyclic heteroaryl wherein the |
| | monocyclic aryl or heteroaryl containing 1-5 heteroatoms selected from the |
| | group consisting of oxygen, nitrogen, and sulfur is optionally substituted |
| | with substituents selected from the group consisting of halogen, hydroxy, |
| | NH₂, and cyano. |
| Family XIV | 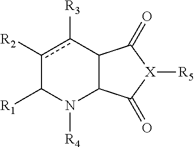 |
| | wherein: |
| | X is optionally oxygen, sulfur, or nitrogen; |
| | [symbol] represents an optional double bond; |
| | R₁-R₃ are independently H, C₁-C₁₄ alkyl, C₂-C₁₄ alkenyl, C₂-C₁₄ alkynyl, |
| | C₃-10 cycloalkenyl, C₄-C₁₄ cycloalkylalkyl, and arylalkyl, each optionally |
| | substituted from 1 to 3 times with substituents selected from the group |
| | consisting of halogen, —OH, cyano, —NH₂, H, C₁-C₁₄ alkyl, C₂-C₁₄ alkenyl, |
| | C₂-C₁₄ alkynyl, C₃-10 cycloalkenyl, and C₄-C₁₄ cycloalkylalkyl, wherein |
| | each C₁-C₁₄ alkyl, C₂-C₁₄ alkenyl, C₂-C₁₄ alkynyl, C₃-10 cycloalkenyl, or |
| | C₄-C₁₄ cycloalkylalky is optionally substituted from 1 to 3 times with |
| | substituents selected from the group consisting of halogen, —OH, cyano, and |
| | —NH₂; |
| | R₄ is optionally H, —NH₂, —NHR₆, —NR₆R₇, wherein R₆ and R₇ are defined as |
| | below; |
| | R₅ is optionally H, C₁-C₁₄ alkyl, C₂-C₁₄ alkenyl, C₂-C₁₄ alkynyl, C₃-C₁₀ |
| | cycloakyl, C₃-C₁₀ cycloalkenyl, and C₄-C₁₄ cycloalkylalkyl, monocyclic |
| | aryl, heterocyclyl, or heteroaryl wherein the heterocyclyl or heteroaryl |
| | contains 1-5 heteroatoms selected from the group consisting of oxygen, |
| | sulfur, and nitrogen, wherein each one of R₅ is optionally substituted with |
| | halogen, —OH, cyano, and —NH₂; |
| | R₆-R₇ are independently monocyclic aryl, heterocyclyl, or heteroaryl |
| | wherein the heterocyclyl or heteroaryl contains 1-5 heteroatoms selected |
| | from the group consisting of oxygen, sulfur, and nitrogen, each of R₆ or R₇ |
| | is optionally substituted with halogen, —OH, cyano, and —NH₂; |
| | with the proviso that when X is oxygen or sulfur R₅ is absent. |
| Family XV | 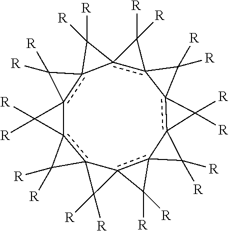 |
| | wherein: |
| | [symbol] represents an optional double bond; and |
| | each R is optionally H, C₁-C₆ alkyl, C₂-C₆ alkenyl, C₂-C₆ alkynyl, C₃-C₆ |
| | cycloalkyl, C₃-C₇ cycloalkylalkyl, —C(O)OR₁, or —OR₂, wherein two R on |
| | adjacent carbon atoms may optionally combine to form a bond or a 3 to 6- |
| | membered monocyclic heterocycle containing 1-3 heteroatoms selected |
| | from the group consisting of oxygen, nitrogen, and sulfur, optionally |
| | substituted from 1 to 3 times with substituents selected from the group |
| | consisting of H, carbonyl, carbamoyl, C₁-C₆ alkyl and C₂-C₆ alkenyl, |
| | wherein R₂ is defined below; |
| | R₁ is optionally H, C₁-C₆ alkyl, C₂-C₆ alkenyl, C₂-C₆ alkynyl, C₃-C₆ |
| | cycloalkyl, or C₃-C₇ cycloalkylakyl; |
| | R₂ is optionally and independently H, C₁-C₆ alkyl, C₂-C₆ alkenyl, C₂-C₆ |
| | alkynyl, C₃-C₆ cycloalkyl, C₃-C₇ cycloalkylakyl, or C(O)R₃, wherein R₃ is |
| | defined below; |
| | R₃ is optionally and independently H, C₁-C₆ alkyl, C₂-C₆ alkenyl, C₂-C₆ |
| | alkynyl, C₃-C₆ cycloalkyl, or C₃-C₇ cycloalkylakyl, each optionally |
| | substituted from 1 to 3 times with substituents selected from the group |
| | consisting of halogen, hydroxy, —NH₂, and cyano. |
| Family XVI | 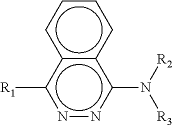 |
| | wherein: |
| | R₁ is optionally and independently H, C₁-C₆ alkyl, C₂-C₆ alkenyl, C₂-C₆ |
| | alkynyl, C₃-C₆ cycloalkyl, C₄-C₇ cycloalkylalkyl, C₁-C₆ alkoxy, mono- or |
| | polycyclic aryl, mono- or polycyclic heterocyclyl, or mono- or polycyclic |
| | heteroaryl, wherein the mono- or polycyclic heterocyclyl or heteroaryl |
| | contains 1-5 heteroatoms selected from the group consisting of oxygen, |
| | nitrogen, and sulfur, wherein each of R₁ are optionally substituted with |
| | substitutents selected from the group consisiting of H, C₁-C₆ alkyl, C₂-C₆ |
| | alkenyl, C₂-C₆ alkynyl, C₃-C₆ cycloalkyl, C₄-C₇ cycloalkylalkyl, R₄S—, |
| | R₄SO₂—, R₄NHSO₂—, and R₄R₅NSO₂—, wherein R₄ and R₅ are defined as |
| | below; |
| | R₂ and R₃ are independently H, C₁-C₆ alkyl, C₂-C₆ alkenyl, C₂-C₆ alkynyl, |
| | C₃-C₆ cycloalkyl, C₄-C₇ cycloalkylalkyl, C₁-C₆ alkoxy, each optionally |
| | substituted from 1 to 3 with substituents selected from the group consisting |
| | of halogen, hydroxy, —NH₂ cyano, C₁-C₆ alkyl, C₂-C₆ alkenyl, C₂-C₆ |
| | alkynyl, C₃-C₆ cycloalkyl, C₄-C₇ cycloalkylalkyl, and C₁-C₆ alkoxy; |
| | or R₂ and R₃ can combine to form a monocyclic heterocyclyl or heteroaryl |
| | containing 1 to 5 heteroatoms selected from the group consisting of |
| | nitrogen, sulfur, and oxygen, each substituted with substituents selected |
| | from the group consisting of hydroxy, —NH₂ cyano, C₁-C₆ alkyl, C₂-C₆ |
| | alkenyl, C₂-C₆ alkynyl, C₃-C₆ cycloalkyl, C₄-C₇ cycloalkylalkyl, and C₁-C₆ |
| | alkoxy; |
| | R₄-R₅ are optionally and independently H, C₁-C₆ alkyl, C₂-C₆ alkenyl, C₂-C₆ |
| | alkynyl, C₃-C₆ cycloalkyl, C₄-C₇ cycloalkylalkyl, C₁-C₆ alkoxy. |
| Family XVII | 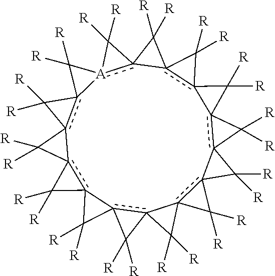 |
| | wherein: |
| | A is a carbon, oxygen, or nitrogen; and |
| | [symbol] represents an optional double bond; |
| | each R is optionally and independently H, C₁-C₆ alkyl, C₂-C₆ alkenyl, C₂-C₆ |
| | alkynyl, C₃-C₆ cycloalkyl, C₄-C₇ cycloalkylalkyl, ═O, OR₁, or two R on a |
| | common carbon atom or on two adjacent carbon atoms may form a cyclic |
| | ether or epoxide; |
| | R₁ are optionally and independently H, C₁-C₆ alkyl, C₂-C₆ alkenyl, C₂-C₆ |
| | alkynyl, C₃-C₆ cycloalkyl, C₄-C₇ cycloalkylalkyl, C₂-C₆ alkoxy, monocyclic |
| | heterocyclyl, or monocyclic heteroaryl wherein the heterocyclyl or |
| | heteroaryl contains 1-5 heteroatoms selected from the group consisting of |
| | oxygen, nitrogen, and sulfur, wherein R is optionally substituted from 1 to 3 |
| | times with substituents selected from the group consisting of C₁-C₆ alkyl, |
| | —OR₃, —NHR₃, and —NR₃R₄, wherein R₃ and R₄ are defined as below; |
| | R₃ and R₄ are optionally and independently H, C₁-C₆ alkyl, C₂-C₆ alkenyl, |
| | C₂-C₆ alkynyl, C₃-C₆ cycloalkyl, C₄-C₇ cycloalkylalkyl; |
| | with the following provisos that (1) when A is oxygen, R is absent; (2) |
| | when A is nitrogen and [symbol] is a double bond, R is absent. |

Examples of suitable compounds of Family I are compounds which have the following structures:

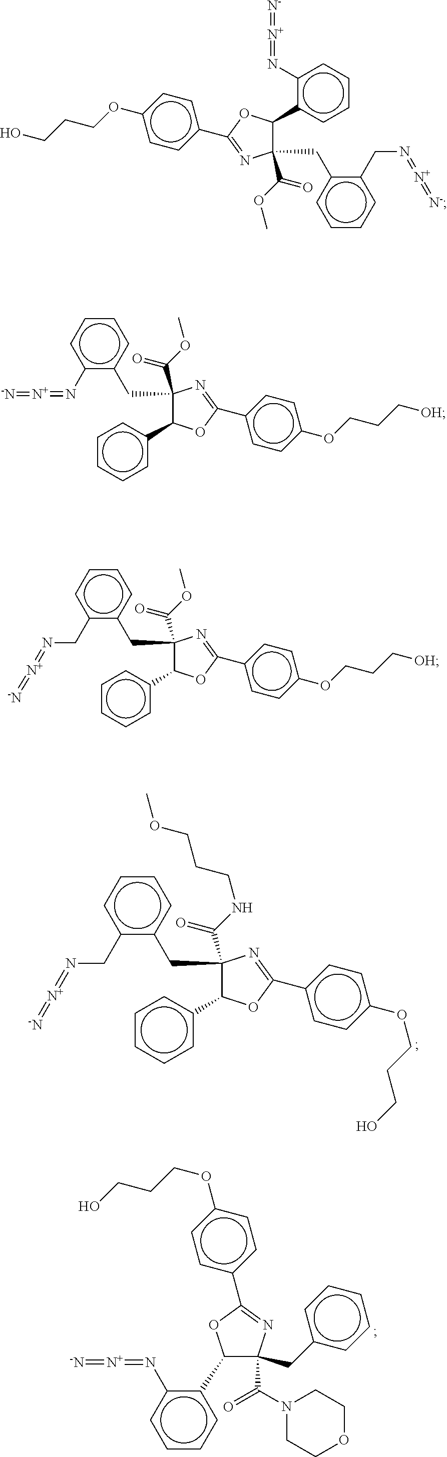

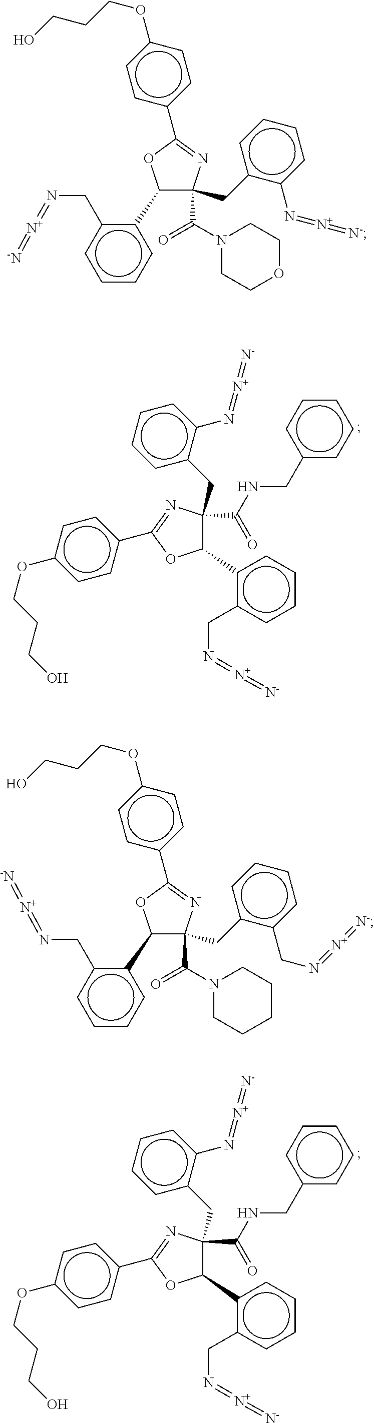

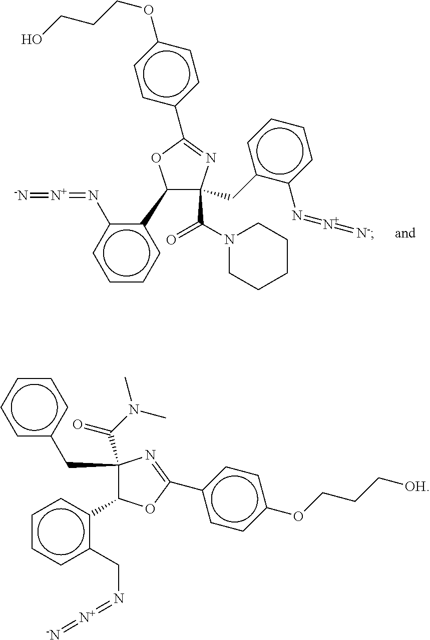

Examples of suitable compounds of Family II are compounds which have the following structures:

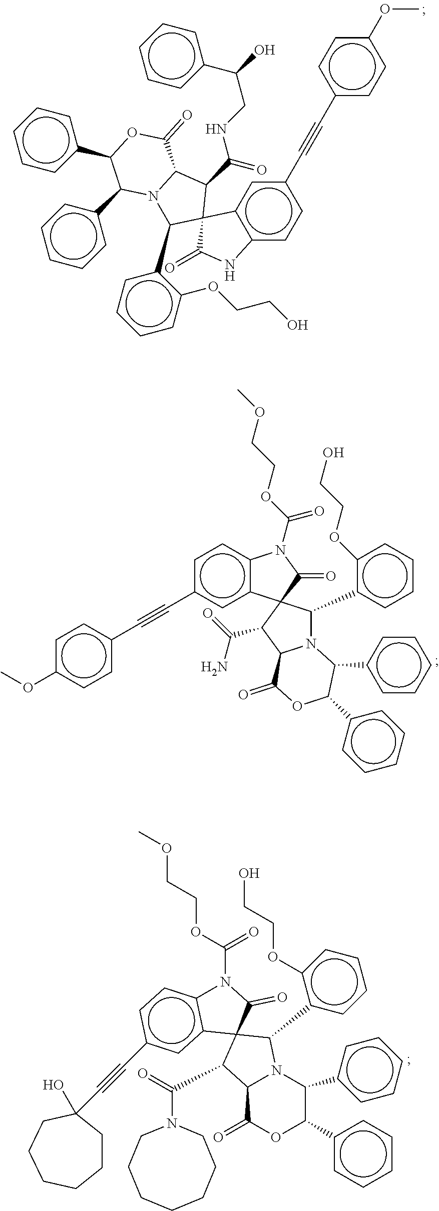

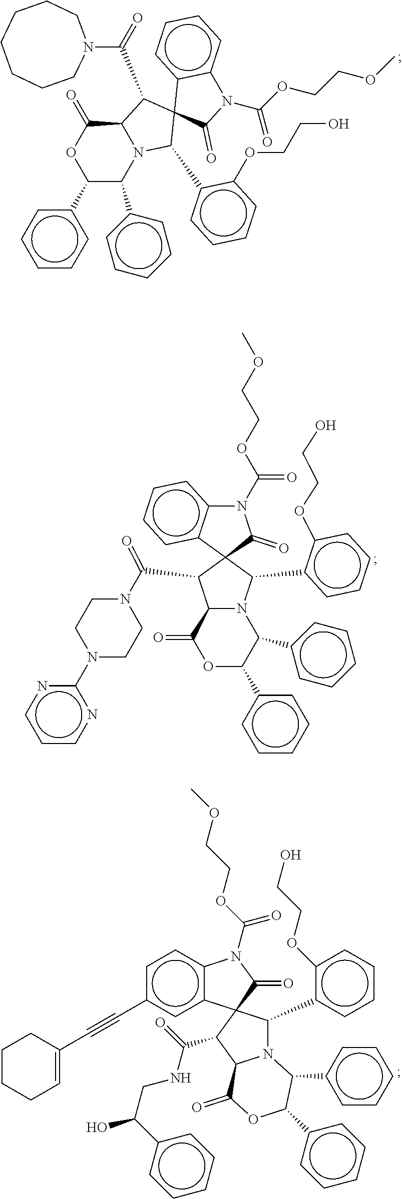

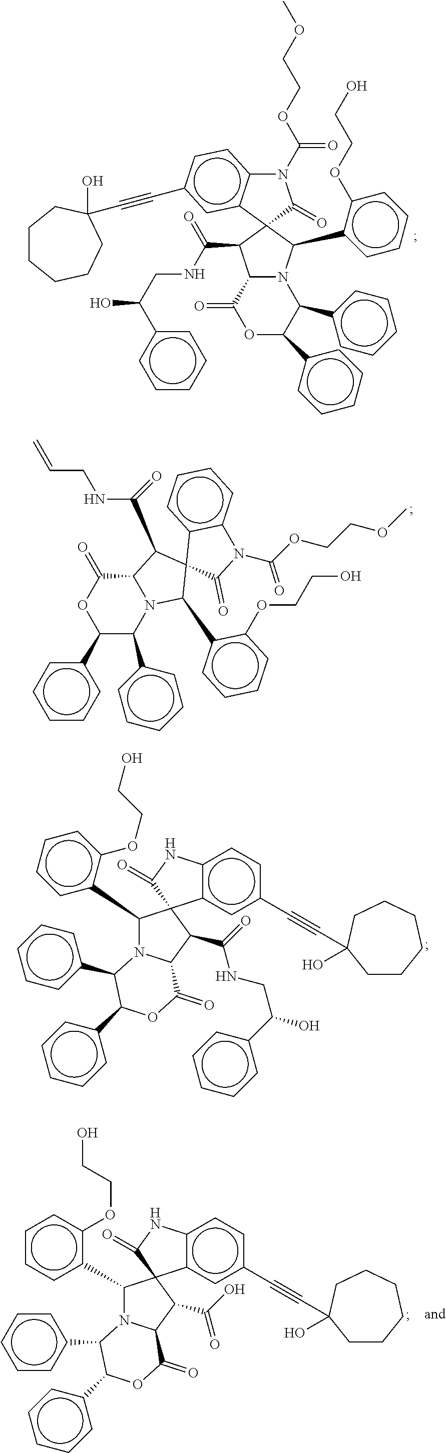

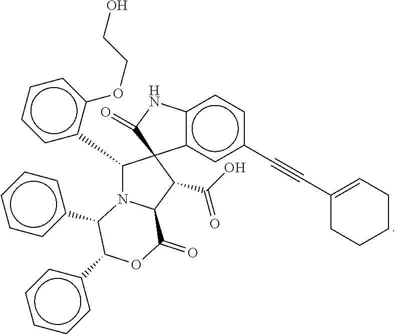

Examples of suitable compounds of Family III are compounds which have the following structures:

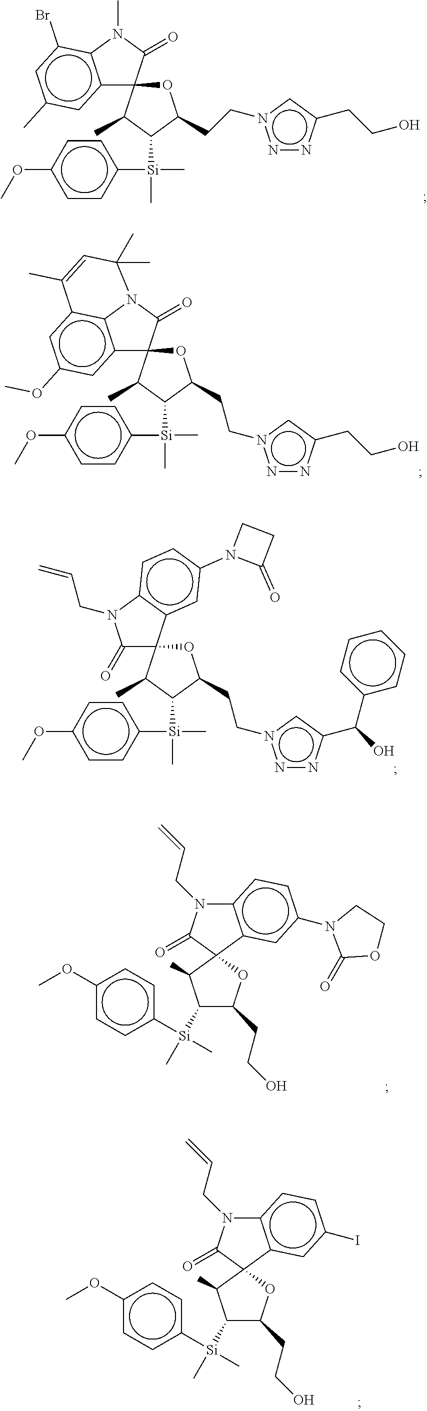

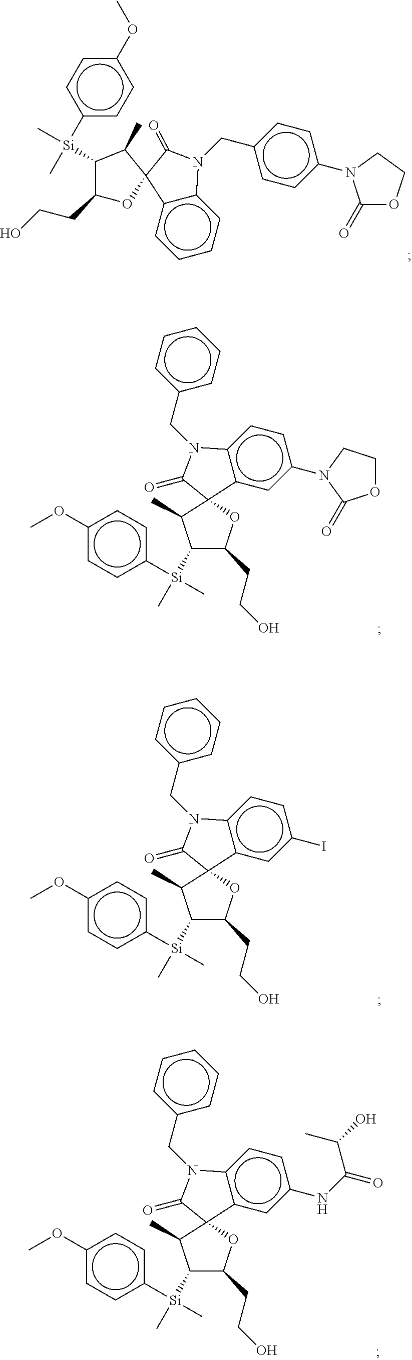

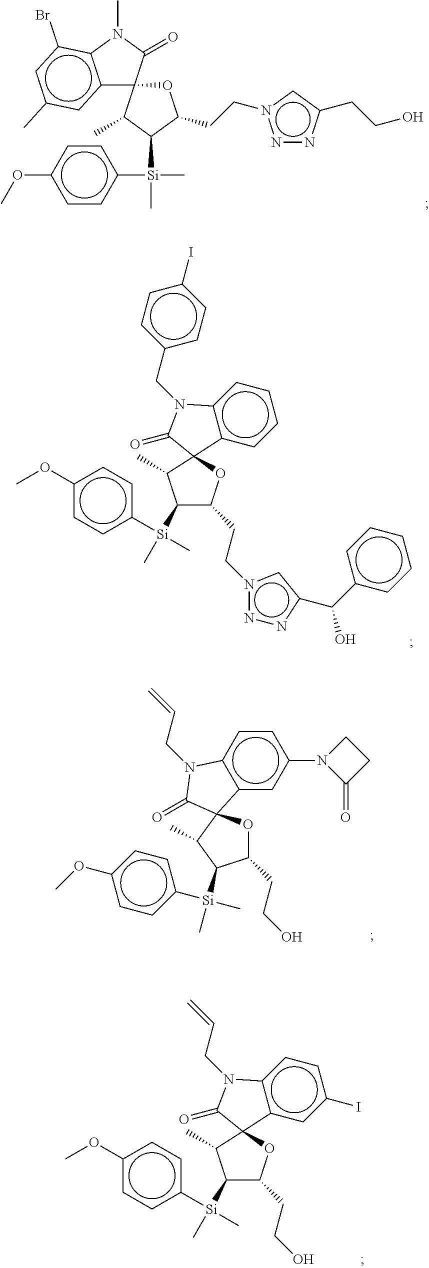

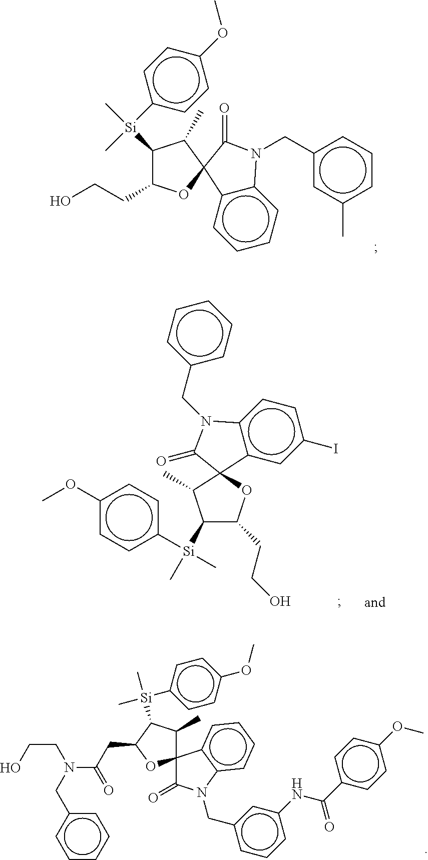

Examples of suitable compounds of Family IV are compounds which have the following structures:

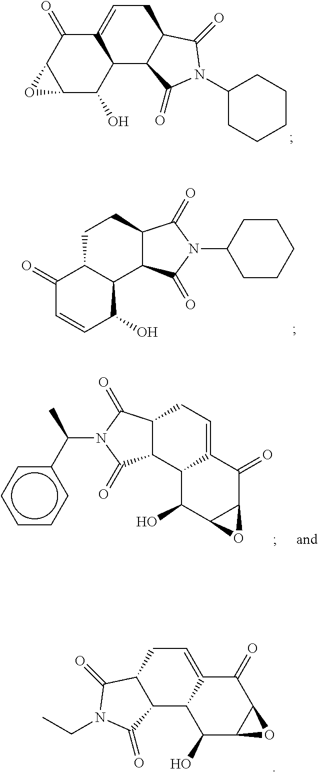

Examples of suitable compounds of Family V are compounds which have the following structures:

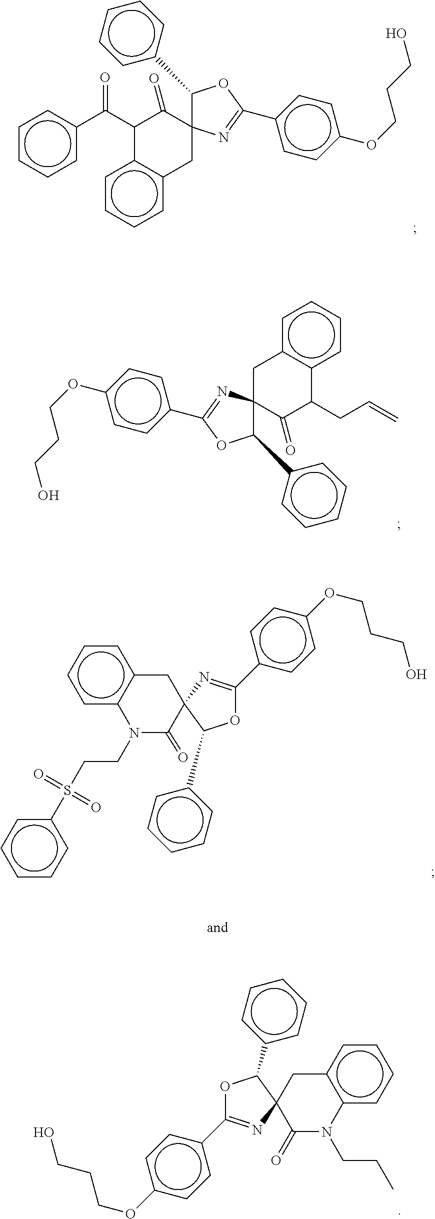

Examples of suitable compounds of Family VI are compounds which have the following structures:

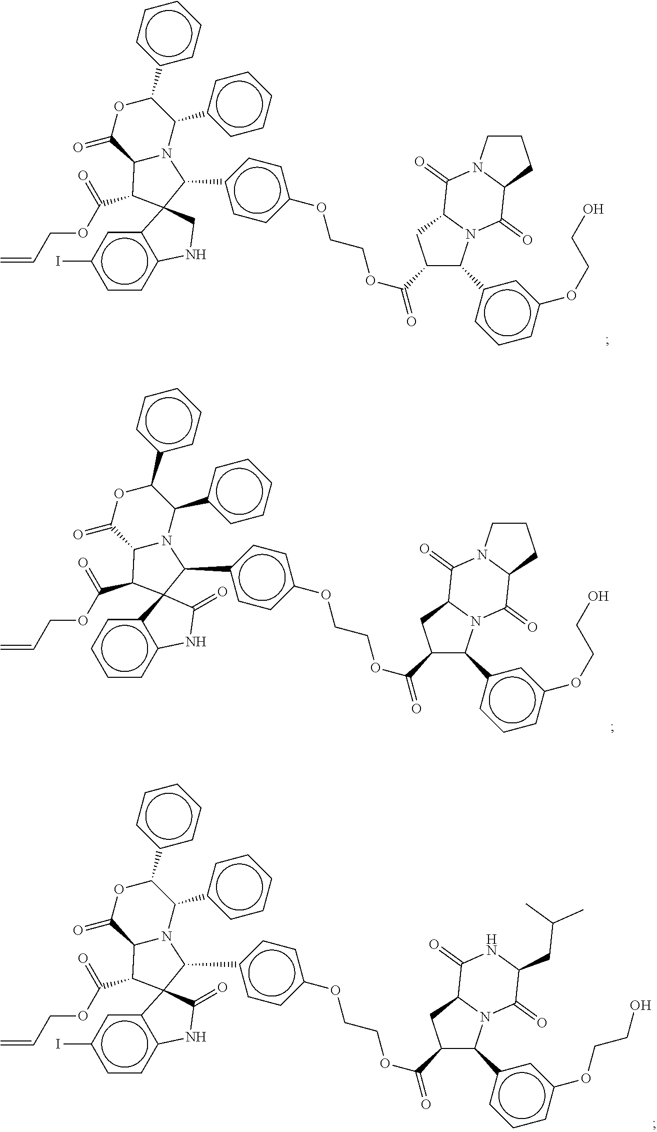

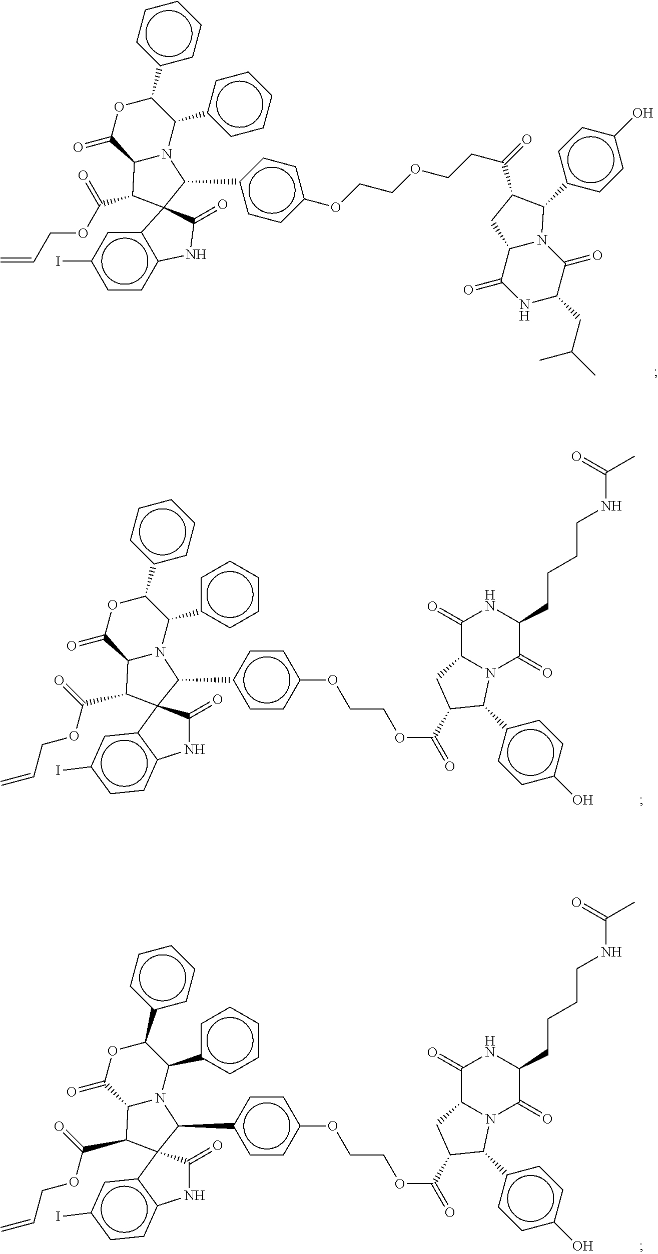

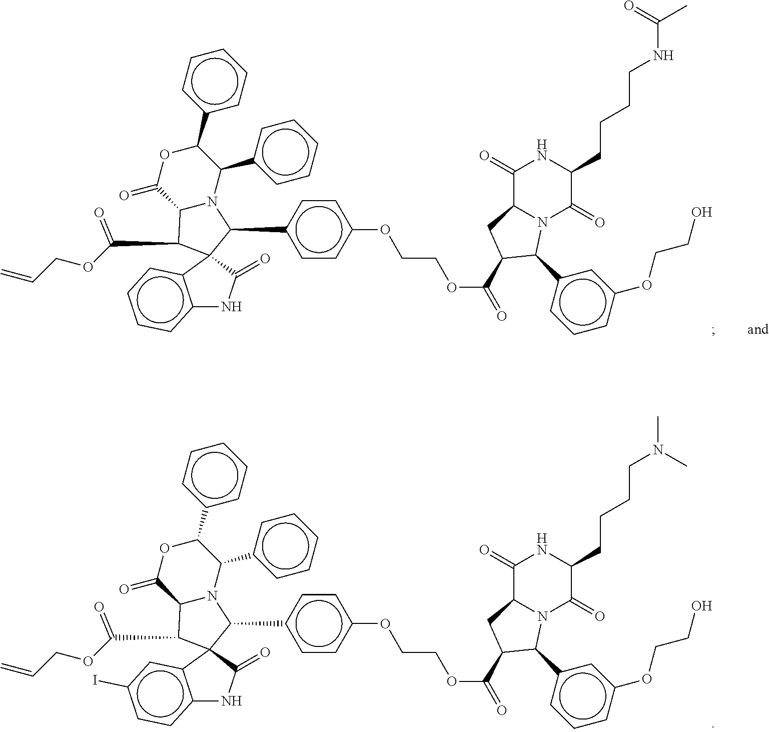

Examples of a suitable compounds of Family VII are compounds which have the following structures:

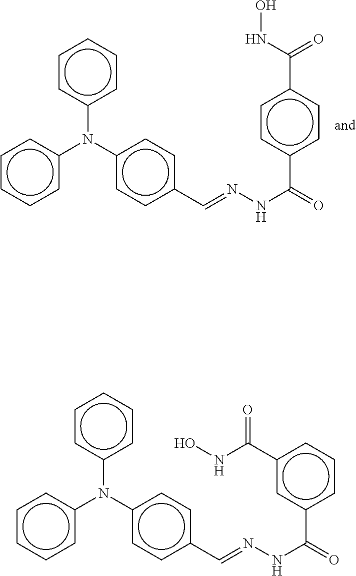

Examples of suitable compounds of Family VIII are compounds which have the following structures:

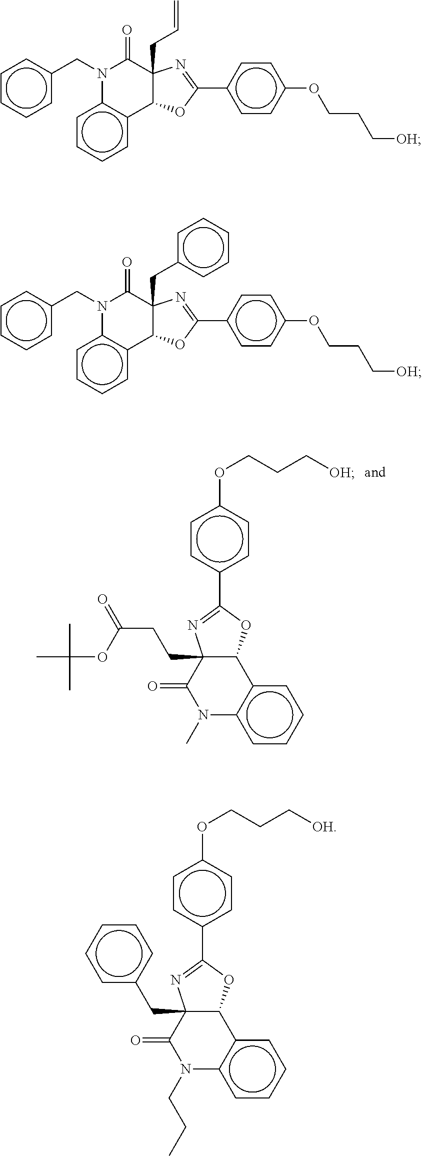

Examples of suitable compounds of Family IX are compounds which have the following structures:

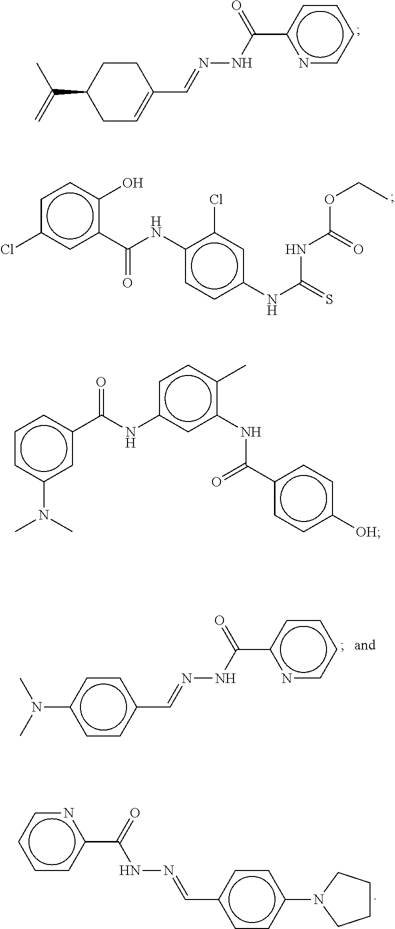

Examples of a suitable compounds of Family X are compounds which have the following structures:

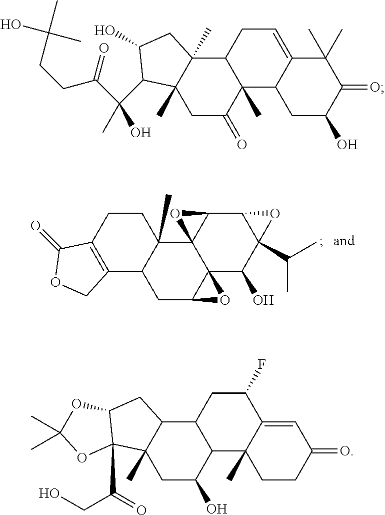

Examples of suitable compounds of Family XI are compounds which have the following structures:

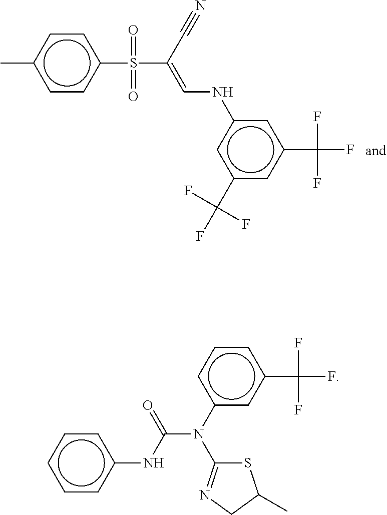

Examples of suitable compounds of Family XII are compounds which have the following structures:

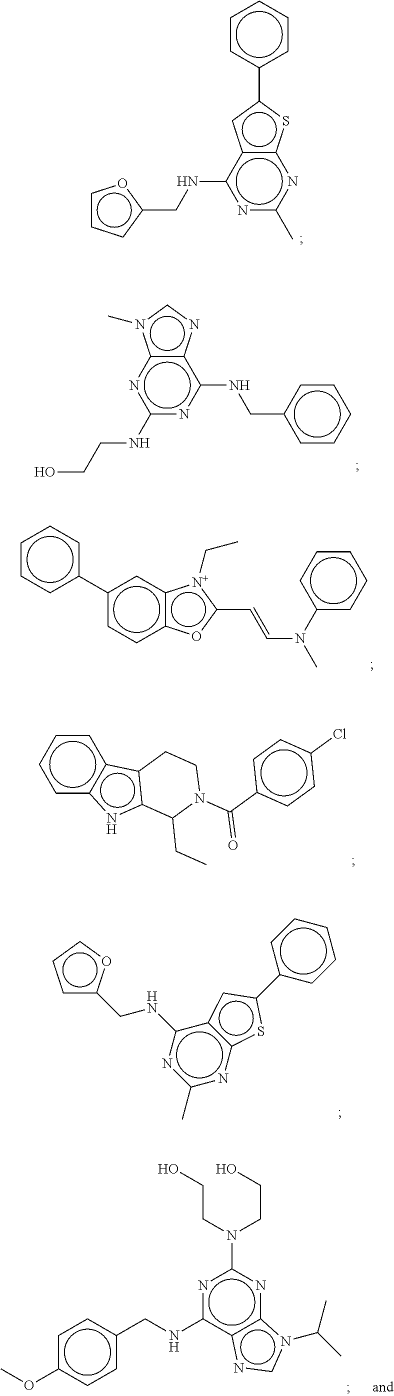

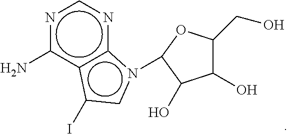

Examples of suitable compounds of Family XIII are compound which have the following structures:

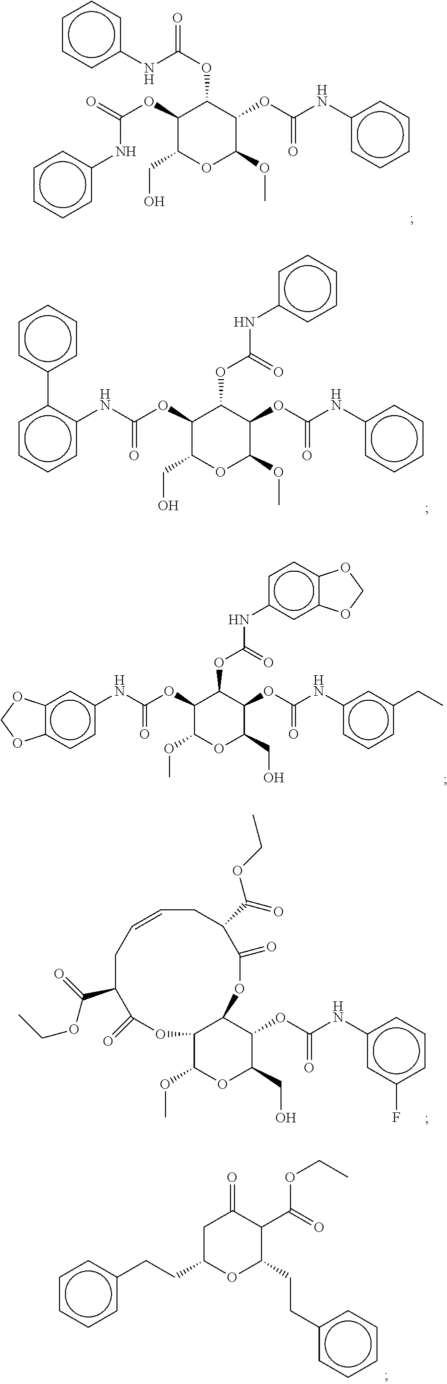

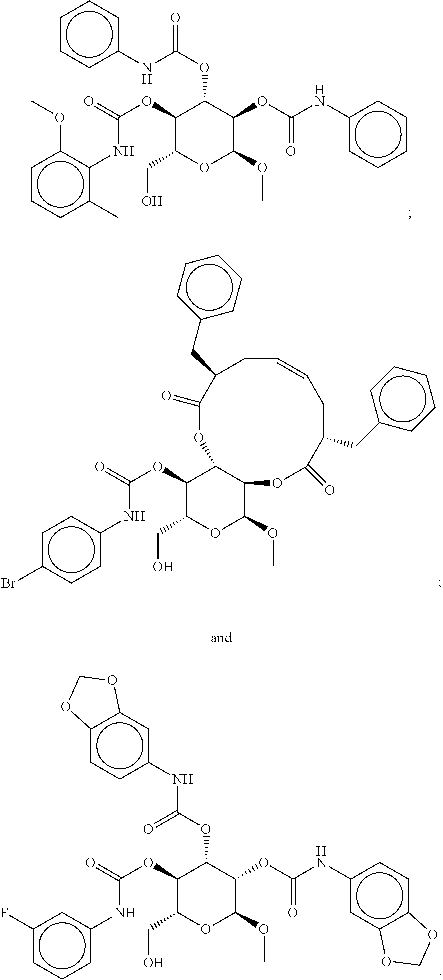

Examples of suitable compounds of Family XIV are compounds which have the following structures:

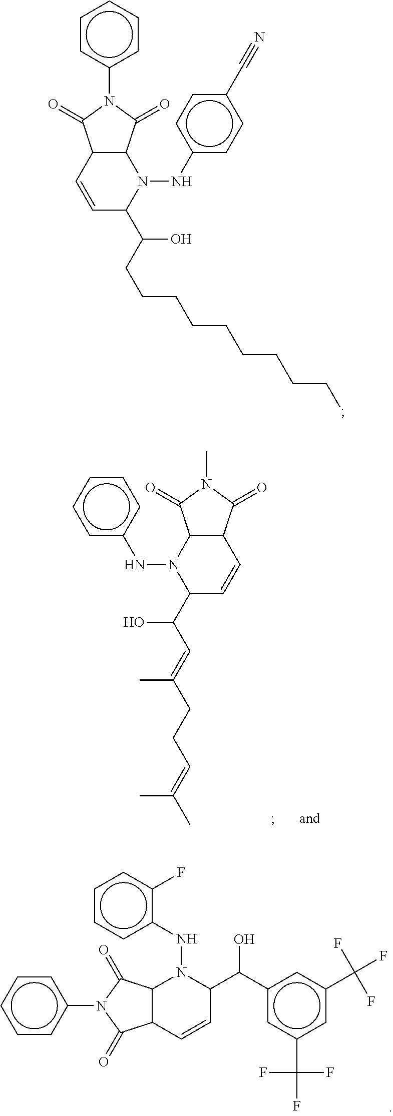

Examples of suitable compounds of Family XV are compounds which have the following structures:

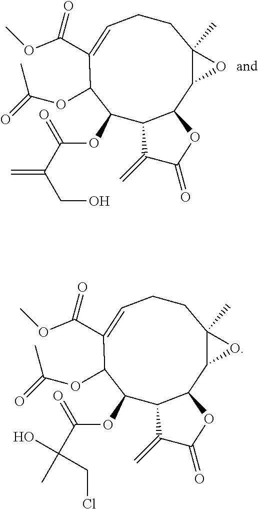

Examples of suitable compounds of Family XVI are compounds which have the following structures:

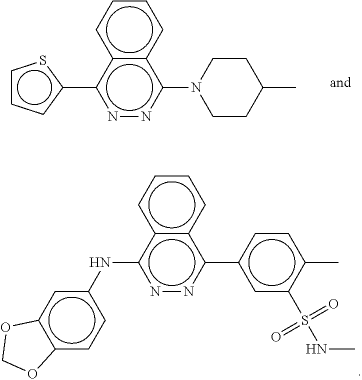

Examples of suitable compounds of Family XVII are compounds which have the following structures:

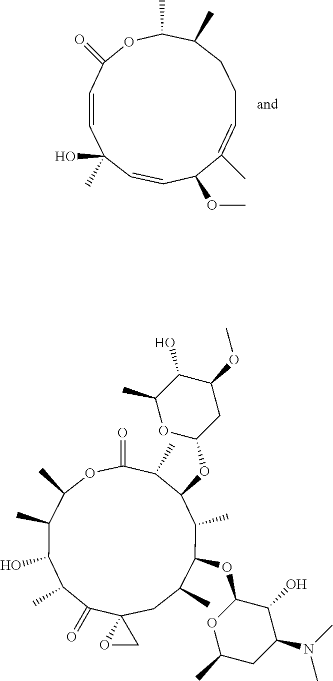

TABLE 2
Other Suitable Compounds

| Compounds | Structures |
|---|---|
| Compound 1 | 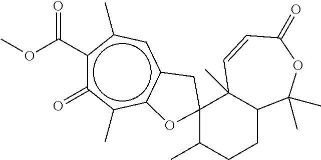 |
| Compound 2 | 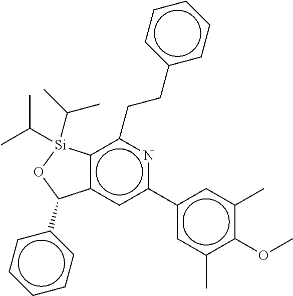 |
| Compound 3 | 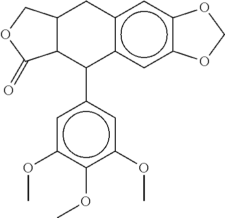 |
| Compound 4 | 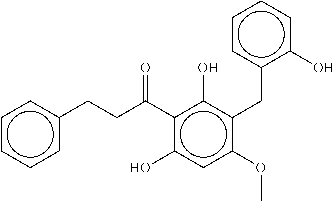 |
| Compound 5 | 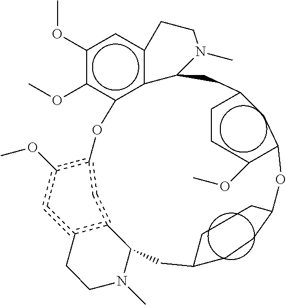 |
| Compound 6 | 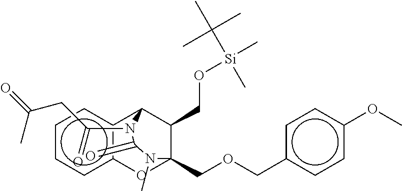 |
| Compound 7 | 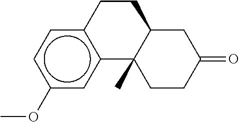 |
| Compound 8 | 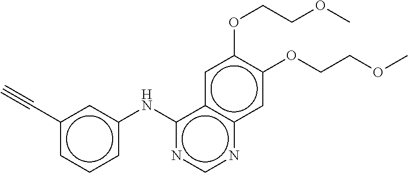 |
| Compound 9 | 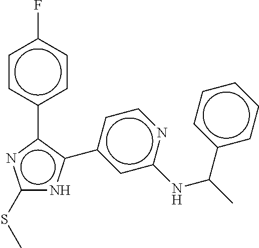 |
| Compound 10 | 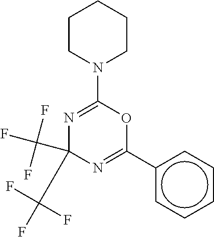 |
| Compound 11 | 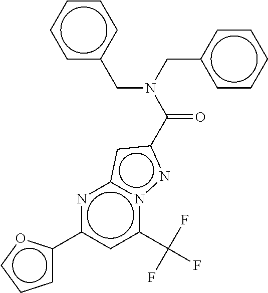 |
| Compound 12 | 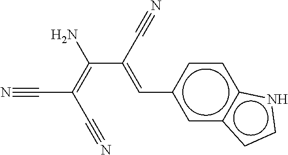 |
| Compound 13 | 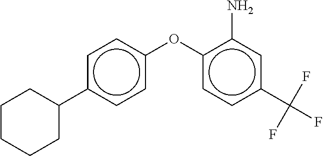 |
| Compound 14 | 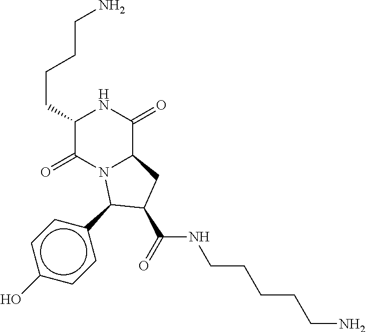 |
| Compound 15 | 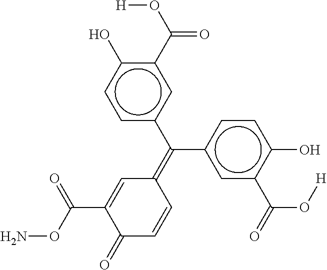 |
| Compound 16 | 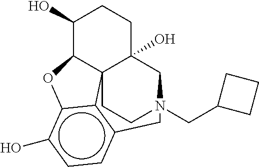 |
| Compound 17 | 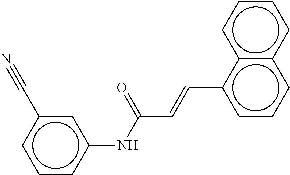 |
| Compound 18 | 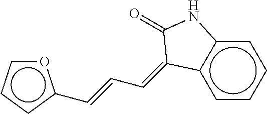 |
| Compound 19 | 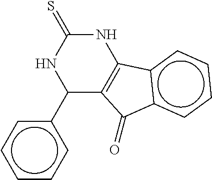 |
| Compound 20 | 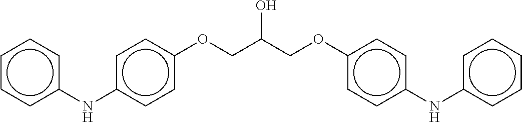 |
| Compound 21 | 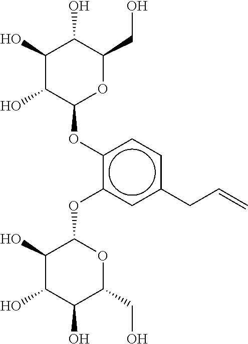 |
| Compound 22 | 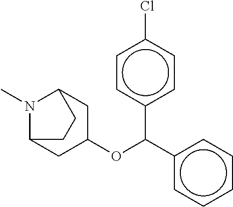 |
| Compound 23 | 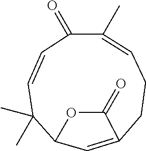 |
| Compound 24 | 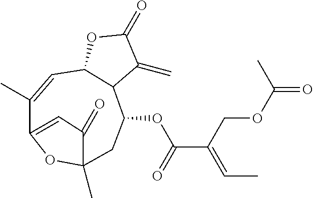 |
| Compound 25 | 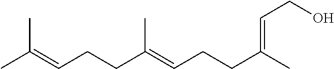 |
| Compound 26 | 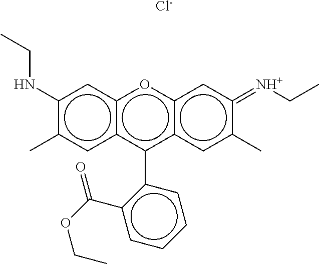 |
| Compound 27 | 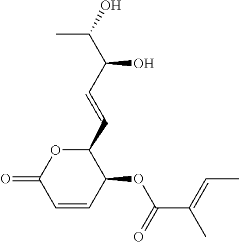 |
| Compound 28 | 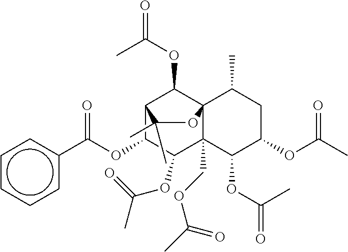 |
| Compound 29 | 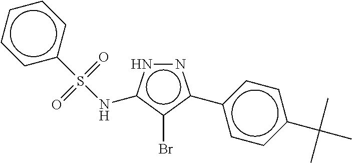 |
| Compound 30 | 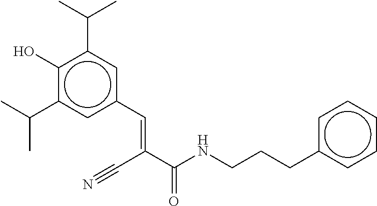 |
| Compound 31 | 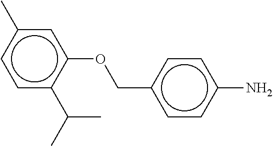 |
| Compound 32 | 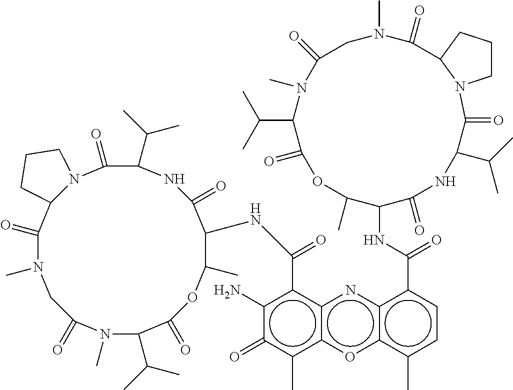 |
| Compound 33 | 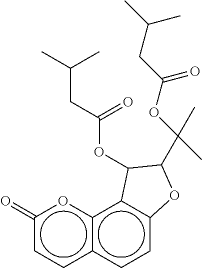 |
| Compound 34 | 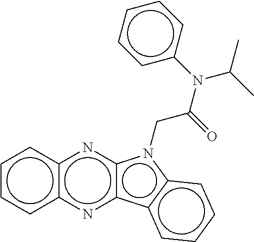 |
| Compound 35 | 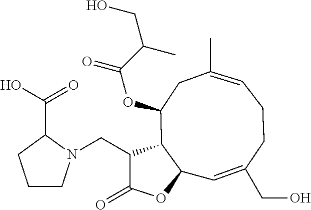 |
| Compound 36 | 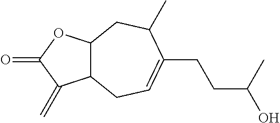 |
| Compound 37 | 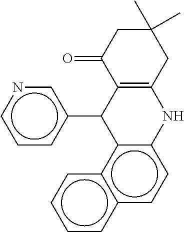 |
| Compound 38 | 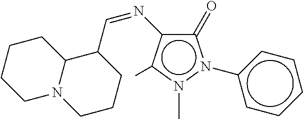 |
| Compound 39 | 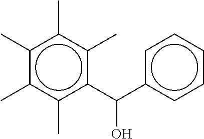 |
| Compound 40 | 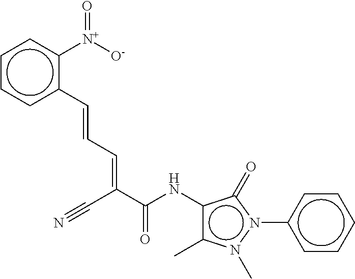 |
| Compound 41 | 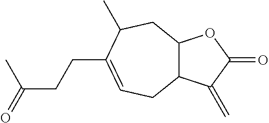 |
| Compound 42 | 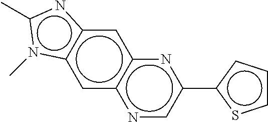 |
| Compound 43 | 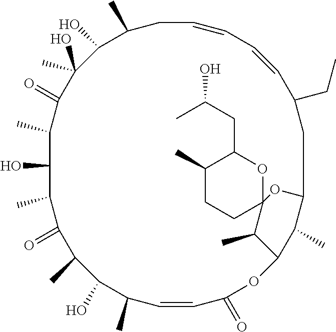 |
| Compound 44 | 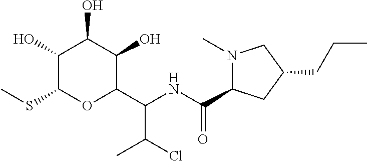 |
| Compound 45 | 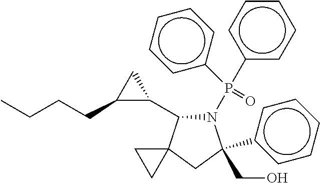 |
| Compound 46 | 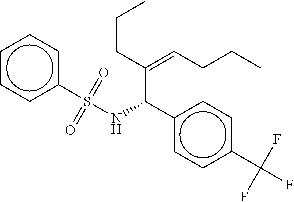 |
| Compound 47 | 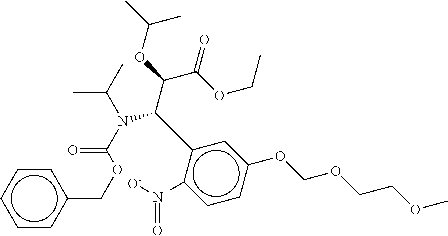 |
| Compound 48 | 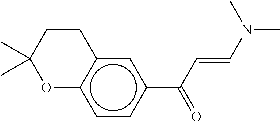 |

The compounds of the present invention can be administered orally, parenterally, for example, subcutaneously, intravenously, intramuscularly, intraperitoneally, by intranasal instillation, by inhalation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assailable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds of the present invention may also be administered directly to the airways in the form of a dry powder. For use as a dry powder, the compounds of the present invention may be administered by use of an inhaler. Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the composition is delivered to the patient. A dry powder inhaler is a system operable with a source of pressurized air to produce dry powder particles of a pharmaceutical composition that is compacted into a very small volume. For inhalation, the system has a plurality of chambers or blisters each containing a single dose of the pharmaceutical composition and a select element for releasing a single dose.

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants and co-solvents and may be filled into conventional aerosol containers that are closed by a suitable metering valve.

### EXAMPLES

### Example 1

### Cell Lines

B16 murine melanoma cells expressing firefly luciferase were used as the parental line for experiments described herein (Murakami et al., Cancer Res. 62:7328 (2002), which is hereby incorporated by reference in its entirety). Human melanoma UACC 1273 and M92047 cell lines are as described in Bittner et al., Nature 406:536 (2000), which is hereby incorporated by reference in its entirety). The human melanoma cell lines Me1375, A2058, Mel 29.6 and Me1501 were obtained from Fred Hutchinson Cancer Research Institute; Seattle, Wash. The murine cell line HT22, a subclone of the HT4 hippocampal cell line, was obtained from The Salk Institute for Biological Studies. Sequences for human WNT3A and WNT5A were amplified by polymerase chain reaction (PCR) and cloned into third generation lentiviral vectors derived from backbone vectors (Dull et al., J. Virol. 72:8463 (1998), which is hereby incorporated by reference in its entirety). These lentiviral vectors contained an EF 1-alpha promoter driving a bi-cistronic message encoding human Wnt isoforms plus GFP. Cells were sorted by fluorescence activated cell sorting (FACS) for GFP expression, with the goal of obtaining cells with approximately equivalent levels of GFP expression.

### Example 2

### Cell Culture

B16 murine melanoma cells were cultured in Dulbeccos modified Eagle's media (DMEM) supplemented with 2% Fetal Bovine Serum, and 1% antibiotic/antimycotic (Invitrogen; Grand Island, N.Y.) (Murakami et al., Cancer Res. 62:7328 (2002), which is hereby incorporated by reference in its entirety). The human melanoma lines Me1375, M92047, A2058, Mel 29.6, Mel501 and Me1526 were cultured in DMEM supplemented with 2% FBS and 1% antibiotic/antimycotic. UACC 1273 cells were cultured in RPMI (Invitrogen; Grand Island, N.Y.) supplemented with 2% FBS and 1% antibiotic/antimycotic. All cell lines were cultured in the presence of 0.02% Plasmocin (InvivoGen; San Diego, Calif.). Synthetic siRNAs (Invitrogen; Grand Island, N.Y.) were transfected into cultured cells at a final concentration of 20 nM using Lipofectamine 2000 (Invitrogen; Grand Island, N.Y.). HT22 cells were cultured in DMEM supplemented with 10% PBS and 1% antibiotic/antimycotic. Sequences for β-catenin siRNA are described in FIG. 8.

### Example 3

### RNA Purification From B16 Melanoma Cells and PCR Analysis

Cells were cultured for approximately 72 hours until they reached 80-90% confluency. RNA was purified using the RNeasy kit using the manufacturer's protocol (Qiagen; Maryland, MD). cDNA was synthesized using Superscript Reverse Transcriptase (Invitrogen; Grand Island, N.Y.). Light Cycler FastStart DNA Master SYBR Green1 (Roche; Mannheim, Germany) was used for real-time PCR as previously described (Major et al., Science 316:1043 (2007), which is hereby incorporated by reference in its entirety). Quantitative PCR results presented in the manuscript are representative of experiments performed on a minimum of three biologic replicates.

### Example 4

### In viva Tumor Inoculation and Measurements of Lymph Node Metastasis

Footpad injections of transduced B16 melanoma cells and measurement of popliteal lymph node and lung metastasis was performed as previously described (Murakami et al., Cancer Res. 62:7328 (2002), which is hereby incorporated by reference in its entirety). All animal studies were performed using IACUC protocols approved by institutional review boards.

### Example 5

### Cell Proliferation Assays

For cell counts by hematocytometer, cells were seeded at a uniform density (usually between 10,000 to 25,000 cells per well) in a 12 or 24 well tissue culture plate in the appropriate media. At the end of 3-7 days, cells were trypsinized, resuspended in the appropriate media and counted. Dead cells were identified by 0.4% Trypan Blue stain and excluded from hematocytometer measurements. Cell proliferation experiments were performed with a minimum of six biologic replicates. Similar results were observed for all cell lines using the MTT assay (ATCC; Manassas, Va.), performed according to manufacturer's protocol. For relative cell proliferation assays of B16: GFP cells incubated with lithium chloride or sodium chloride, cell proliferation was measured by luciferase assay. Cell cycle analysis was performed using DAPI-staining and flow cytometry. The Ki-67 rabbit monoclonal antibody was purchased from ThermoFisher (catalog no. RM-9106).

### Example 6

### Immunohistochemistry and Immunoblotting Studies

A polyclonal rabbit anti-β-catenin antibody was used for detection of β-catenin (1:1000 dilution for immunoblot, 1:200 dilution for immunohistochemistry). Cells were grown on 18 mm glass coverslips, for 48-72 hours, fixed using 4% paraformaldehyde, permeabilized using 0.25% Triton X-100, and then blocked with 10% goat serum. Goat anti-rabbit Alexa Fluor-568 antibody (Molecular Probes; Eugene, Oreg.) was diluted 1:1000. Cells were counterstained for nucleic acid with DAPI (Molecular Probes; Eugene, Oreg.). Paraffin-embedded nevus sections were stained using an antibody dilution of 1:200. Cellular lysates were obtained by lysing cells on plate with a 0.1% NP-40 based buffer and analyzed by NuPage 4-12% gradient gels (Invitrogen; Grand Island, N.Y.). The WNT5A antibody was obtained from Cell Signaling Technologies (Danvers, Mass.).

### Example 7

### Tumor Microarrays

Tumor microarrays were assembled at the Yale Tissue Microarray Facility. Staining and scoring of tissue microarrays was performed using automated quantification (AQUA) as previously described (Camp et al., Nat. Med. 8:1323 (2002), which is hereby incorporated by reference in its entirety). Statistical analysis, including Kaplan-Meier survival probabilities, ANOVA, and t-tests, was performed using the GraphPad Prism software package (GraphPad Software; La Jolla, Calif.).

### Example 8

### cDNA Microarrays

Agilent whole mouse genome array analysis was performed through the microarray core facility at the Huntsman Cancer Institute (Salt Lake City, Utah). Data analysis, including the t-test (Pan, Bioinformatics 18:546 (2002), which is hereby incorporated by reference in its entirety) was performed using the TM4 microarray software suite, which is freely available online (Saeed et al., Biotechniques 34:374 (2003), which is hereby incorporated by reference in its entirety). Two-channel hybridizations were performed with labeled cDNA isolated from three biologic replicates each for cells expressing either WNT3A or WNT5A, using cDNA from GFP-expressing cells as the reference sample. These studies revealed gene sets regulated in both WNT3A and WNT5A cells (FIG. 8), which were then filtered to obtain the top 10% of most variant genes in the WNT3A and WNT5A datasets. Subsequently, an unpaired two-tailed t-test analysis was used to identify genes that were significantly different between the most variant genes in the WNT3A and WNT5A replicate samples, using an arbitrary p-value of p<0.04 as a cut-off. The rationale for further comparing the regulated genes in WNT3A cells to those in WNT5A cells was based on the finding that WNT5A did not have significant phenotypic effects (pigmentation, proliferation or cell cycle), and this subsequent comparison allowed identification of potentially important genes regulated by WNT3A that might be missed by setting arbitrary cut-off values for significant genes (i.e. 2-fold upregulated or 50% downregulated).

### Example 9

### High Throughput Small Molecule Screen

Compounds were dissolved in dimethylsulphoxide (DMSO). For the primary screen, performed in duplicate, HT22 cells stably expressing the beta-catenin activated reporter (BAR) were cultured in growth medium (DMEM/10% FBS/1% antibiotic). 3000 cells per well were transferred to 384-well clear bottom plates (Nalgene Nunc; Rochester, N.Y.) in 30 μl of growth medium. The following day, 100 nL of compound and 10 μL of either growth media or WNT3A conditioned media (E.C.₅₀ dose) was transferred to the cells. The next day each well was imaged using transmitted light with the ImageXpress Micro (Molecular devices; Sunnyvale, Calif.) followed by the addition of 10 μL, of Steady-Glo (Promega; Madison, Wis.) as per the manufacture's instructions, and luminescence measurement on an EnVision Multilabel plate reader (PerkinElmer; Waltham, Mass.). Viability was scored by analyzing the ImageXpress images. As described in detail in Seiler et al. (Seiler et al., Nucleic Acids Res. 36:D351 (2008), which is hereby incorporated by reference in its entirety), each compound well received an algebraically signed Z-score corresponding to the number of standard deviations it fell above or below the mean of a well-defined mock-treatment distribution of DMSO controls. Z-score normalized data from the growth media stimulus group were sorted by average percent change. The fold-increase over the background of DMSO controls for each treatment was also calculated. The top 50 compounds with the greatest percent change of activity with the growth media were then resorted based on the percent change with the WNT3A stimulus.

### Example 10

### Nuclear β-catenin Correlates With Improved Patient Survival

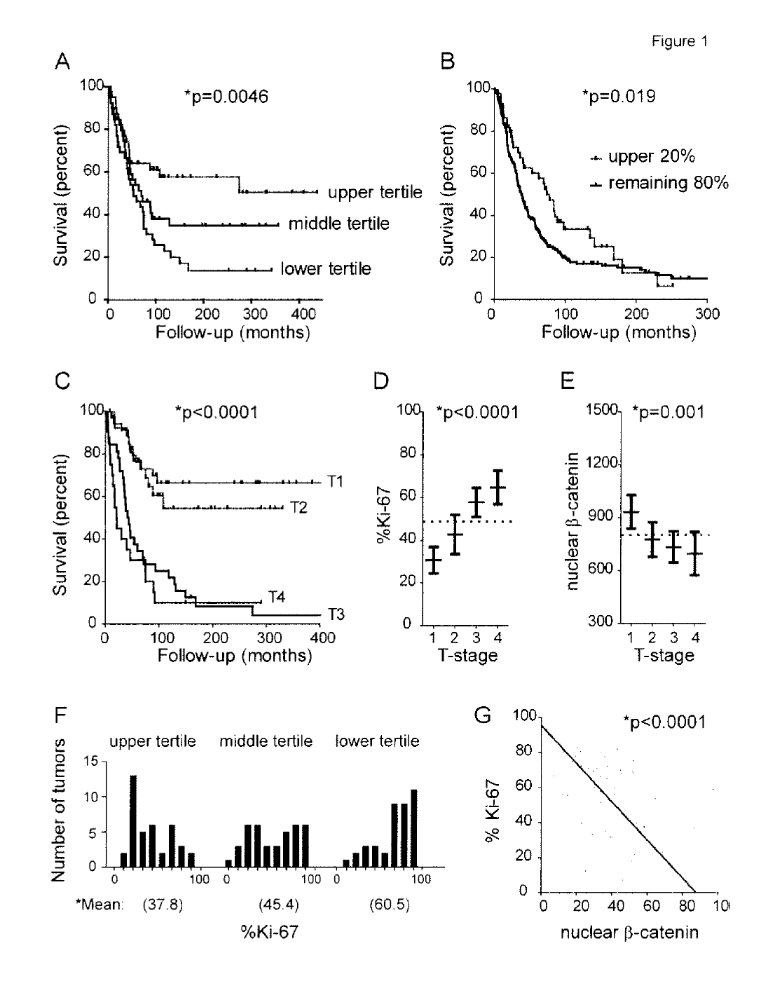

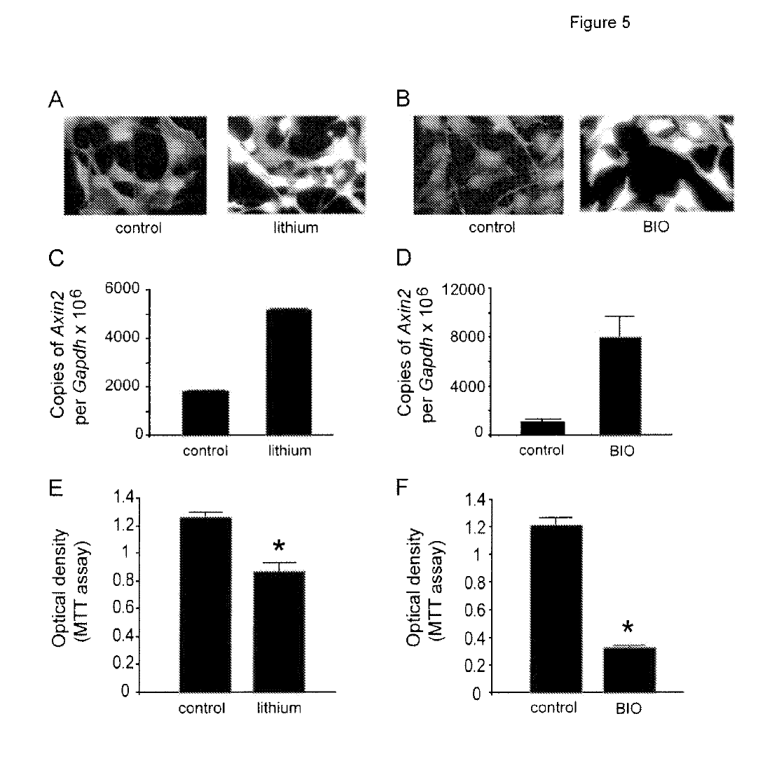

Using the expression of nuclear β-catenin as a clinical surrogate marker for Wnt/P-catenin activation (Bachmann et al., Clin. Cancer Res. 11:8606 (2005); T. Kageshita et al., Br. J. Dermatol. 145:210 (2001); Maelandsmo et al., Clin. Cancer Res. 9:3383 (2003), which are hereby incorporated by reference in their entirety), a tumor microarray composed of 343 cores (118 primary tumors, 225 metastases) from patient tumors (Camp et al., Nat. Med. 8:1323 (2002), which is hereby incorporated by reference in its entirety) was scored. Survival probabilities for patients were estimated using Kaplan-Meier analysis after stratifying primary tumors into tertiles based on nuclear 3-catenin expression (FIG. 5). This analysis reveals that higher expression of nuclear β-catenin in both primary tumors (FIG. 1A) and in metastases and recurrences (FIG. 1B) predicts significantly increased patient survival. Also, levels of nuclear β-catenin are lower in metastases and recurrences compared to primary tumors (FIG. 5). These findings confirm and extend previous reports of improved prognosis with elevated nuclear β-catenin in melanoma (Bachmann et al., Clin. Cancer Res. 11:8606 (2005); T. Kageshita et al., Br. J. Dermatol. 145:210 (2001); Maelandsmo et al., Clin. Cancer Res. 9:3383 (2003), which are hereby incorporated by reference in their entirety).

### Example 11

### Nuclear β-catenin is Negatively Correlated with Proliferation

As tumor depth measurements (Breslow thickness) were obtained for 113 primary tumors in our array cohort, this sub-group of patients was analyzed based on the Breslow thickness stratification used as reported (Thompson, J. A., Semin. Oncol. 29:361 (2002), which is hereby incorporated by reference in its entirety). Increasing tumor depth is correlated with a lower probability of survival (FIG. 1C) and with a higher degree of proliferation, which is measured by the percentage of cells expressing Ki-67 (FIG. 1D). By contrast, nuclear β-catenin levels are highest for shallow tumors (T1)) and decrease significantly with increased tumor depth (FIG. 1E).

The percentage of tumors staining positive is then analyzed for the cellular proliferative marker Ki-67 (% Ki-67). Strikingly, distribution histograms of % Ki-67 staining in primary tumors stratified by expression of nuclear β-catenin show a statistically significant shift towards increased proliferation (elevated % Ki-67 staining) in the groups with lower nuclear β-catenin (FIG. 1F). It is shown that there is no correlation between expression of α-catenin and % Ki-67 staining, and PCNA is used as an independent marker of proliferation (FIG. 5). Taken together these data demonstrate that elevated nuclear B-catenin is negatively associated with proliferation as measured by either tumor size/depth, or by the markers Ki-67 and PCNA.

### Example 12

### Activation of Wnt/β-catenin Signaling Changes Melanoma Cell Fate

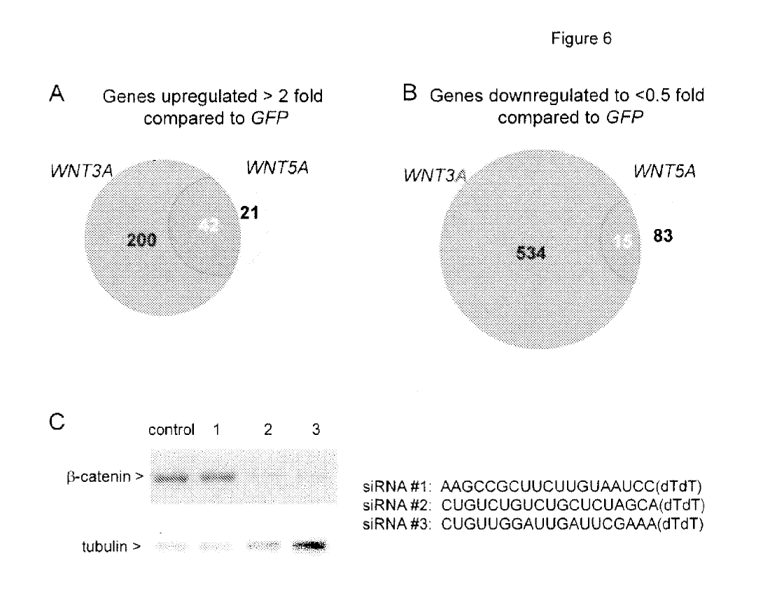

Wnts, which can activate or antagonize B-catenin signaling, were investigated in order to elicit changes in melanoma cells cultured in vitro that might be consistent with the above clinical data. Since melanoma tumors appear to express WNT3A (FIG. 6), which has a pivotal role in the regulation of melanocyte biology (Dorsky et al., Genes Dev. 14:158 (2000); Fang et al., Stem Cells 24:1668 (2006), which are hereby incorporated by reference in their entirety), and they express WNT5A, which is elevated in melanoma metastases (Bittner et al., Nature 406:536 (2000); Weeraratna et al., Oncogene 23:2264 (2004), which are hereby incorporated by reference in their entirety), B16 mouse melanoma cells were transduced with lentivirus constructs encoding WNT3A, WNT5A, or a GFP control.

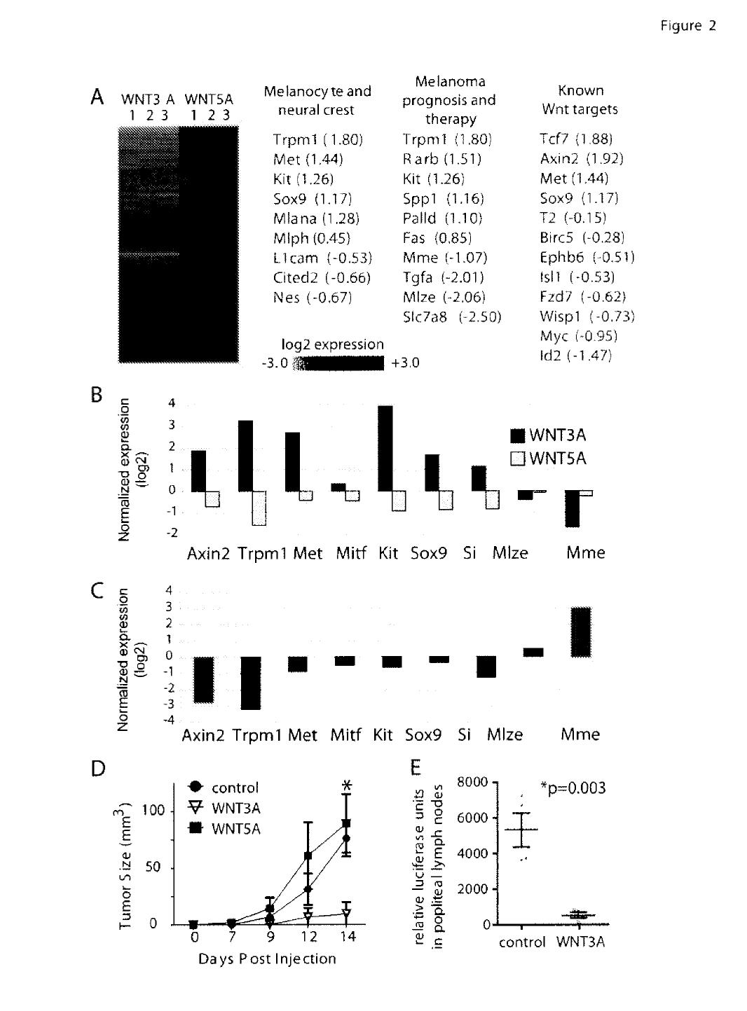

B16:WNT3A cells exhibit strikingly increased pigmentation compared to GFP or WNT5A cells (FIG. 2A). Scoring cells for nuclear accumulation of β-catenin revealed that only cells expressing WNT3A, and not WNT5A or GFP, exhibit elevated β-catenin (FIG. 2C). As a positive control, it was shown that conditioned media (CM) from B16 cells expressing WNT3A activates a β-catenin-responsive reporter in UACC1273 melanoma cells (FIG. 2D), confirming that these cells were secreting active WNT3A. Also, it was shown that B16 cells expressing WNT3A exhibit marked increases in expression of the β-catenin target gene Axin2 (Jho et al., Mol. Cell Biol. 22:1172 (2002), which is hereby incorporated by reference in its entirety) compared to B16:GFP cells (FIG. 2E).

In vitro cell proliferation studies using the MTT cell proliferation assay showed that B16 cells expressing WNT3A exhibit decreased proliferation compared to cells expressing GFP or WNT5A (FIG. 2F). This finding was paralleled in human cell lines (FIG. 6). Cell cycle profiles were then compared to the Wnt-transduced melanoma cell lines, and found that cells expressing WNT3A exhibit an increased population in G1, with a decreased population in S phase, compared to control cells (FIG. 2G). Together, these data suggest that WNT3A can induce differentiation of the melanoma cells to a cell fate that is more pigmented and less proliferative.

### Example 13

### Elevation of Melanocyte Differentiation Markers by WNT3A

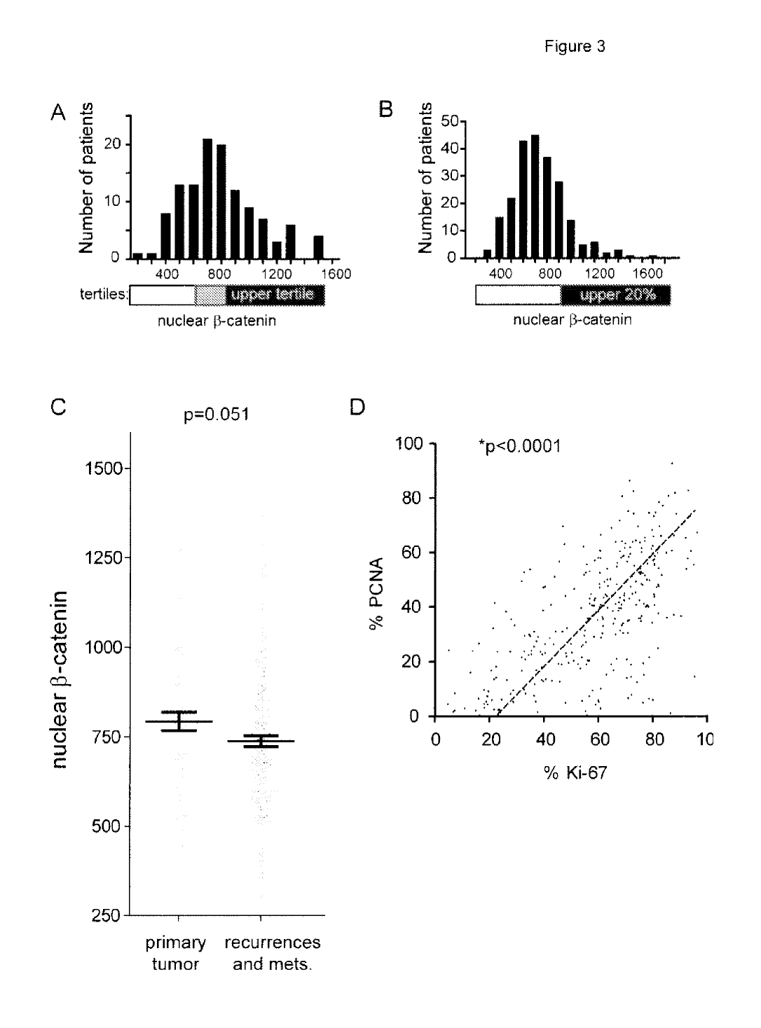

Next, a genome-wide transcriptional profiling was performed to gain further insights into the consequences of expression of WNT3A and WNT5A, which revealed that levels of transcripts elevated by WNT3A were actually reduced by WNT5A (FIG. 3B). Among the most highly significant genes elevated by WNT3A (FIG. 3 A) are Axin2 (Jho et al., Mol. Cell Biol. 22:1172 (2002), which is hereby incorporated by reference in its entirety) and Tcf7 (Roose et al., Science 285:1923 (1999), which is hereby incorporated by reference in its entirety), which are direct targets of Wnt/β-catenin signaling; Mme and Mlze, downregulated genes previously linked to melanoma progression (Watabe et al., Jpn. J. Cancer Res. 92:140 (2001); Bilalovic et al., Mod. Pathol. 17:1251 (2004), which are hereby incorporated by reference in their entirety); Mitf, linked to pigment cell fate, and Trpm1, Met, Sox9 and Kit, which are highly expressed during melanocyte and neural crest development (Loftus et al., Proc. Natl. Acad. Sci. USA 96:9277 (1999, which is hereby incorporated by reference in its entirety)). To confirm the array data levels of selected transcripts were measured by quantitative PCR (FIG. 3B). To establish that the effects of WNT3A on gene expression were specific, it was demonstrated that the changes in gene expression were antagonized by β-catenin siRNA (FIG. 3C). The transcriptional profiling thus supports the conclusion, evident from visual examination of cells (FIG. 2A), that WNT3A promotes melanoma cells adopting characteristics of melanocyte differentiation.

### Example 14

### WNT3A Reduces Melanoma Tumor Size and Metastasis in Mice

While expression of Trpm1 was elevated by WNT3A (FIG. 3B), its expression is usually reduced during melanoma progression. Taken with the observed changes in cell fate and proliferation seen in cells expressing WNT3A, this led to the prediction that cells expressing WNT3A would form less proliferative and less aggressive tumors in vivo. Indeed, implantation of WNT3A-transduced B16 cells into the footpads of C57BL/6 mice, significantly decreased tumor growth compared to B16 cells transduced with GFP or WNT5A (FIG. 3D) and decreased metastases to popliteal lymph nodes (FIG. 3E).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating a subject for a condition mediated by the Wnt/β-catenin pathway, said method comprising:
selecting a subject with a condition mediated by the Wnt/β-catenin pathway and administering to the selected subject a compound selected from the group consisting of those set forth in Table 1, or Table 2, and a pharmaceutically acceptable salt thereof.

2. The method of claim 1, wherein the subject is human.

3. The method of claim 1, wherein a compound of Table 1 or a pharmaceutically acceptable salt thereof is administered.

4. The method of claim 1, wherein a compound of Table 2 or a pharmaceutically acceptable salt thereof is administered.

5. (canceled)

6. A method of inhibiting the Wnt/β-catenin pathway in a subject comprising:
selecting a subject in need of Wnt/β-catenin pathway inhibiting and
administering to the selected subject a compound selected from the group consisting of those set forth in Table 1, or Table 2, and a pharmaceutically acceptable salt thereof.

7. The method of claim 6, wherein a compound of Table 1 or a pharmaceutically acceptable salt thereof is administered.

8. The method of claim 6, wherein a compound of Table 2 or a pharmaceutically acceptable salt thereof is administered.
